(19) United States
(12) Patent Application Publication
(10) Pub. No.: US 2021/0395698 A1
(43) Pub. Date: Dec. 23, 2021

(54) METHODS AND COMPOSITIONS FOR CULTIVATING PLURIPOTENT CELL SUSPENSIONS

(71) Applicant: Life Technologies Corporation, Carlsbad, CA (US)

(72) Inventors: Rhonda NEWMAN, Carlsbad, CA (US); Michael AKENHEAD, Carlsbad, CA (US)

(21) Appl. No.: 17/354,281

(22) Filed: Jun. 22, 2021

Related U.S. Application Data
(60) Provisional application No. 63/042,419, filed on Jun. 22, 2020.

(51) Int. Cl. C12N 5/074

(52) U.S. Cl. CPC C12N 5/0696; C12N 2501/727; C12N 2501/235; C12N 2501/115; C12N 2501/33; C12N 2501/998; C12N 2501/15; C12N 2533/90

(57) ABSTRACT

Described herein are cell culture media and/or cell culture media supplements that comprise at least one GSK3 inhibitor, a ROCK inhibitor, and one or more mitogenic growth factors, and methods of culturing and/or expanding pluripotent stem cells in 3D culture formats in the compositions described herein.

### RELATED APPLICATIONS

This application claims the benefit of priority to U.S. Provisional Patent Application No. 63/042,419 filed on Jun. 22, 2020, the entire contents of which is incorporated herein by reference.

### TECHNICAL FIELD

Described herein are cell culture media or supplements that comprise at least one small molecule inhibitor, mitogenic growth factor, and albumin, salts thereof, esters thereof, or combinations thereof. The supplement can be added to media or a cell culture at various times to increase pluripotent stem cell (PSC) growth, enhance PSC proliferation, maintain PSC pluripotency, maintain spheroid morphology, maintain PSC morphology, increase PSC passage count, increase PSC culture scale, as compared to customary PSC media.

### BACKGROUND

Human pluripotent stem cell (hPSC) expansion enables generation of a nearly unlimited pool of cells for use in downstream differentiation, disease modeling, drug discovery, and therapeutic applications. Furthermore, appropriate proliferation and differentiation of PSCs can potentially be used to generate an unlimited source of cells suited for transplantation to treat diseases that result from cell damage or dysfunction.

However, many of the reported methods for growing PSCs are suboptimal. For example, murine embryonic stem cells are often maintained in an undifferentiated state using feeder-free cultures supplemented with leukemia inhibitory factor (LIF). Human embryonic stem cells (hESCs) differentiate when the cells are cultured without a feeder cell layer or conditioned medium from a suitable feeder cell line, even in the presence of LIF. Systems which employ feeder cells (or conditioned media from feeder cell cultures) typically use cells from a different species than that of the stem cells being cultivated, e.g., mouse embryonic fibroblasts (MEF) form the feeder layer in most reported undifferentiated growth of hESCs. Moreover, reports of feeder-free systems typically require the use of conditioned medium from MEF cultures, which does not cure the need for non-xenogeneic products/agents. Even systems that employ human feeder cells have the drawback of exposing the undifferentiated cells to undefined culture conditions, and therefore, many stem cell culture conditions are often not reproducible. Additionally, for many applications, including cell therapies, large quantities of PSCs are required. Over the past decade, pluripotent stem cell culture has migrated to the use of feeder-free culture media systems and specialized matrices to support their expansion under adherent monolayer conditions. While these culture systems have greatly simplified the workflow, the expansion potential is limited by surface area and scale-out in culture flasks requires significant hands on time and increased risk of culture contamination. For example, the number of PSCs required for islet transplantation using the Edmonton protocol is estimated to be on the order of 10⁹ to 10¹⁰ cells/patient.

Cell culture media provide the nutrients for maintaining or growing cells in a controlled in vitro environment. The characteristics and compositions of the cell culture media vary depending on the particular cellular requirements and any functions for which the cells are cultured. Media is typically manufactured as dry powders, liquids, liquid concentrates, agglomerated media, or agglomerated media pellets. See e.g., U.S. Pat. Nos. 6,383,810 and 6,627,426 and U.S. Pat. App. Pub. Nos. US 2018/0142203 A1 and US 2019/004831 A1, each of which are incorporated by reference herein for teachings related to cell culture media.

There is a need for methods and compositions for PSC culture, stabilization, and large-scale production; wherein defined cell culture media or supplements increase PSC growth, proliferation, passage count, culture scale, and maintain pluripotency and morphology in cultured PSCs.

### SUMMARY

One embodiment described herein is a pluripotent stem cell (PSC) composition comprising: a cell culture basal medium; one or more mitogenic growth factors; and a first inhibitor comprising a glycogen synthase kinase-3 (GSK3) inhibitor, salts thereof, or esters thereof; and a second inhibitor comprising a rho kinase (ROCK) inhibitor, salts thereof, or esters thereof; wherein the composition provides one or more of enhancing PSC growth, enhancing PSC proliferation, maintaining PSC pluripotency, maintaining spheroid morphology, maintaining PSC morphology, increasing PSC passage count, or increasing PSC culture scale, as compared to customary PSC media. In one aspect, the composition further comprises a second small molecule inhibitor, comprising a rho kinase inhibitor, salts thereof, or esters thereof. In embodiments, the GSK3 inhibitor and/or the ROCK inhibitor is provided in a cell culture supplement. In some embodiments, the GSK3 inhibitor and/or the ROCK inhibitor is provided in the basal medium.

In one aspect, the GSK3 inhibitor is selected from the group consisting of: CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), BIO ((2′Z,3′E)-6-bromoindirubin-3′-oxime), AR-A 014418 (N-[(4-methoxyphenyl)methyl]-N′-(5-nitro-2-thiazolyl)urea)0, Kenpaullone (9-bromo-7,12-dihydro-indolo[3,2-d][1]benzazepin-6(5H)-one), SB 216763 (dichlorophenyl)-4-(1-methyl-1H-indol-3-yl)-1H-pyrrole-2,5-dione), or SB 415286 (3-[(3-chloro-4-hydroxyphenyl)amino]-4-(2-nitrophenyl)-1H-pyrrole-2,5-dione), their salts, their esters, and combinations thereof. In another aspect, the GSK3 inhibitor is CHIR99021. In one aspect, the ROCK inhibitor is selected from the group consisting of: Y-27632 ((R)-(+)-trans-4-(1-aminoethyl)-N-(4-pyridyl)cyclohexanecarboxamide), Chroman 1, Emricasan, Polyamines, Trans-ISRIB, thiazovavin, and combinations thereof. In another aspect, the ROCK inhibitor is Y-27632 ((R)-(+)-trans-4-(1-aminoethyl)-N-(4-pyridyl)cyclohexanecarboxamide). In another aspect, the ROCK inhibitor inhibits ROCK1 activity. In another aspect, the ROCK inhibitor inhibits ROCK2 activity. In another aspect, the ROCK inhibitor inhibits ROCK1 activity and ROCK2 activity.

In one aspect, the one or more mitogenic growth factors comprises EGF, TGF-α, TGF-β, bFGF, BDNF, HGF, heregulin (HRG), or KGF, or combinations thereof. In another aspect, the PSC composition comprises at least two mitogenic growth factors and the at least two mitogenic growth factors comprise: EGF and TGF-α, EGF and TGF-β, EGF and bFGF, EGF and BDNF, EGF and HGF, EGF and HRG, EGF and KGF, TGF-α and TGF-β, TGF-α and bFGF, TGF-α and BDNF, TGF-α and HGF, TGF-α and HRG, TGF-α and KGF, TGF-β and bFGF, TGF-β and BDNF, TGF-β and HGF, TGF-β and HRG, TGF-β and KGF, bFGF and BDNF, bFGF and HGF, bFGF and HRG, bFGF and KGF, BDNF and HGF, BDNF and HRG, BDNF and KGF, HGF and HRG, HGF and KGF, or HRG and KGF.

In one aspect the PSC composition further comprises albumin or peptides thereof. In another aspect, the albumin is selected from the group consisting of: human serum albumin, bovine serum albumin, rat serum albumin, mouse serum albumin, horse serum albumin monkey serum albumin, pig serum albumin, a recombinant serum albumin, functional fragments of any of the foregoing, and combinations thereof. In one aspect, the PSC composition further comprises one or more extracellular matrix (ECM) components. In another aspect, the one or more ECM components is selected from the group consisting of: fibronectin, laminin, nidogen, collagen, vitronectin, or heparan sulfate proteoglycans, or functional fragments thereof. In another aspect, the PSC composition further comprises transferrin and/or insulin.

In one embodiment, the PSC composition comprises one or more of: amino acids, carbohydrates, vitamins, minerals, fatty acids, trace elements, antioxidants, salts, nucleosides, buffering agents, surfactants, or combinations thereof. In one aspect, the amino acids comprise one or more of glycine, alanine, arginine, asparagine, aspartic acid, cysteine, cystine, glutamic acid, glutamine, histidine, isoleucine, leucine, lysine, methionine, phenylalanine, proline, serine, threonine, tryptophan, tyrosine, valine, salts thereof, esters thereof, or di- or tri-peptides thereof. In another aspect, the vitamins comprise one or more of biotin (B7), choline, folic acid (B9), niacinamide (B3), pyridoxine (B6), riboflavin (B2), thiamine (B1), cobalamin (B12), inositol, retinol (A), pantothenic acid (B5), ascorbic acid (C), cholecalciferol (D), tocopherol (E), phylloquinone (K), lipoic acid, linoleic acid, para-aminobenzoic acid, salts thereof, esters thereof, or any combination of thereof. In another aspect, the salts comprise one or more salts selected from the group consisting of: calcium chloride, cupric sulfate, ferric nitrate, ferric sulfate, magnesium chloride, magnesium sulfate, potassium chloride, potassium iodide, sodium chloride, sodium phosphate dibasic, sodium phosphate monobasic, zinc sulfate, pyridoxine HCl, sodium, potassium, magnesium, calcium, ammonium, phosphate, carbonate, bicarbonate, sulfate, citrate, acetate, nitrate, ions of any of the foregoing, and any combination thereof. In another aspect, the fatty acids comprise one or more of caprylic acid, capric acid, lauric acid, myristic acid, palmitic acid, stearic acid, arachidonic acid, linoleic acid, linolenic acid, oleic acid, palmitoleic acid, cholesterol synthetic, d/l-tocopherol acetate, behenic acid, lignoceric aid, cerotic acid, myristoleic acid, sapienic acid, elaidic acid, vaccenic acid, α-linolenic acid, erucic acid, eicosapentaenoic acid, or docosahexaenoic acid.

In some embodiments, the PSC composition is serum-free. In other embodiments, the PSC composition is animal origin free. In embodiments, the PSC composition further comprises an induced pluripotent stem cell (iPSC) or an embryonic stem cell (ESC).

Another embodiment described herein is a use of the PSC composition for culturing pluripotent stem cells (PSCs) in suspension culture, wherein the composition provides one or more of enhancing PSC growth, enhancing PSC proliferation, maintaining PSC pluripotency, maintaining spheroid morphology, maintaining PSC morphology, increasing PSC passage count, or increasing PSC culture scale as compared to customary PSC media.

Another embodiment described herein is a method for growing pluripotent stem cells (PSCs) in suspension, the method comprising: providing PSCs to be cultured in suspension; contacting the PSCs with a first composition in a culture vessel to form a suspension culture; and culturing the suspension culture under conditions favorable for PSC expansion, where the the first composition comprises: (a) a cell culture basal medium; (b) a first small molecule inhibitor, comprising a glycogen synthase kinase-3 (GSK3) inhibitor, salts thereof, or esters thereof; (c) a second small molecule inhibitor, comprising a rho kinase inhibitor, salts thereof, or esters thereof; (d) one or more mitogenic growth factors; and optionally (e) one or more albumins or peptides thereof. In one aspect, the pluripotent stem cells are human pluripotent stem cells. In another aspect, the PSC is an induced pluripotent stem cell (iPSC) or an embryonic stem cell (ESC).

In one aspect, the providing step comprises dissociating PSCs to single cells. In another aspect, the PSCs are provided as single cells prior to the contacting step. In one aspect, the contacting is at a seeding step for the suspension culture. In another aspect, the seeding step is at passaging of the suspension culture.

In one embodiment, following contacting and culturing with the first composition, the method further comprises contacting the PSCs with a second composition comprising (a) a cell culture basal medium; (b) a first small molecule inhibitor, comprising a glycogen synthase kinase-3 (GSK3) inhibitor, salts thereof, or esters thereof; (d) one or more mitogenic growth factors; and (e) one or more albumins or peptides thereof, at least one day after the seeding or the passaging step to modify the suspension culture medium; and culturing the suspension culture in the presence of the second composition. In another embodiment, following contacting and culturing with the first composition, the method further comprises contacting the PSCs with a second composition comprising (a) a cell culture basal medium; (d) one or more mitogenic growth factors; and (e) one or more albumins or peptides thereof, at least one day after the seeding or the passaging step to modify the suspension culture medium; and culturing the suspension culture in the presence of the second composition.

In one aspect, the step of contacting with the second composition comprises: (a) replacing the first composition with the second composition; (b) overlaying the second composition on to the suspension culture; or (c) exchanging a portion of the first composition with the second composition. In one aspect, the (c) exchanging a portion of the first composition with the second composition comprises exchanging at least 25% of the first composition with an equivalent amount of the second composition. In another aspect, the (c) exchanging a portion of the first composition with the second composition comprises exchanging at least 50% of the first composition with an equivalent amount of the second composition. In one aspect, the method further comprises exchanging at least 25% of the suspension culture medium with an equivalent amount of fresh second composition at least one day after contacting the cells with the second composition. In another aspect, the method further comprises exchanging at least 50% of the suspension culture medium with an equivalent amount of fresh second composition at least one day after contacting the cells with the second composition. In one aspect, the further exchanging of the suspension culture medium is subsequently performed daily. In another aspect, the further exchanging of the suspension culture medium is subsequently performed every-other-day.

In one aspect, the culture vessel is selected from a multi-well plate, a flask, or a bioreactor. In one aspect, the suspension culture has a volume of at least 20 mL. In another aspect, the suspension culture has a volume of at least 100 mL. In one aspect, the method further comprises agitating the cells while culturing the suspension culture in the first and second compositions. In another aspect, the agitating is at about 30 RPM to about 180 RPM.

In some embodiments, at least one of the first composition and the second composition is serum-free. In other embodiments, at least one of the first composition and the second composition is animal origin free. In embodiments, the first composition, the second composition, or combination thereof provide one or more of enhancing PSC growth, enhancing PSC proliferation, maintaining PSC pluripotency, maintaining spheroid morphology, maintaining PSC morphology, increasing PSC passage count, or increasing PSC culture scale as compared to customary PSC media.

In one aspect, the GSK3 inhibitor of the first composition and/or the second composition is selected from the group consisting of: CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), BIO ((2′Z,3′E)-6-bromoindirubin-3′-oxime), AR-A 014418 (N-[(4-methoxyphenyl)methyl]-N′-(5-nitro-2-thiazolyl)urea)0, Kenpaullone (9-bromo-7,12-dihydro-indolo[3,2-d][1]benzazepin-6(5H)-one), SB 216763 (dichlorophenyl)-4-(1-methyl-1H-indol-3-yl)-1H-pyrrole-2,5-dione), or SB 415286 (3-[(3-chloro-4-hydroxyphenyl)amino]-4-(2-nitrophenyl)-1H-pyrrole-2,5-dione), their salts, their esters, and combinations thereof. In another aspect, the GSK3 inhibitor is CHIR99021. In one aspect, the ROCK inhibitor of the first composition is selected from the group consisting of: Y-27632 ((R)-(+)-trans-4-(1-aminoethyl)-N-(4-pyridyl)cyclohexanecarboxamide), Chroman 1, Emricasan, Polyamines, Trans-ISRIB, thiazovavin, and combinations thereof. In another aspect, the ROCK inhibitor is Y-27632 ((R)-(+)-trans-4-(1-aminoethyl)-N-(4-pyridyl)cyclohexanecarboxamide). In another aspect, the ROCK inhibitor inhibits ROCK1 activity. In another aspect, the ROCK inhibitor inhibits ROCK2 activity. In another aspect, the ROCK inhibitor inhibits ROCK1 activity and ROCK2 activity.

In one aspect, the one or more mitogenic growth factors of the first composition and/or the second composition comprises EGF, TGF-α, TGF-β, bFGF, BDNF, HGF, heregulin (HRG), or KGF, or combinations thereof. In another aspect, the first composition and/or the second composition comprises at least two mitogenic growth factors and the at least two mitogenic growth factors comprise: EGF and TGF-α, EGF and TGF-β, EGF and bFGF, EGF and BDNF, EGF and HGF, EGF and HRG, EGF and KGF, TGF-α and TGF-β, TGF-α and bFGF, TGF-α and BDNF, TGF-α and HGF, TGF-α and HRG, TGF-α and KGF, TGF-β and bFGF, TGF-β and BDNF, TGF-β and HGF, TGF-β and HRG, TGF-β and KGF, bFGF and BDNF, bFGF and HGF, bFGF and HRG, bFGF and KGF, BDNF and HGF, BDNF and HRG, BDNF and KGF, HGF and HRG, HGF and KGF, or HRG and KGF.

In one aspect, the first composition and/or the second composition comprises albumin or peptides thereof, and the albumin is selected from the group consisting of human serum albumin, bovine serum albumin, rat serum albumin, mouse serum albumin, horse serum albumin monkey serum albumin, pig serum albumin, a recombinant serum albumin, functional fragments of any of the foregoing, and combinations thereof. In one aspect, the first composition and/or the second composition further comprises one or more extracellular matrix (ECM) components. In another aspect, the one or more ECM components is selected from the group consisting of: fibronectin, laminin, nidogen, collagen, vitronectin, or heparan sulfate proteoglycans, or functional fragments thereof. In another aspect, the first composition and/or the second composition further comprises transferrin and/or insulin. In one aspect, the first composition and/or the second composition comprises one or more of: amino acids, carbohydrates, vitamins, minerals, fatty acids, trace elements, antioxidants, salts, nucleosides, buffering agents, surfactants, or combinations thereof.

### DESCRIPTION OF THE DRAWINGS

FIG. 1 shows assessment of impact graphs of individual small molecules (CHIR99021, PD0325901, SP00125, BIRD796, and Go6983) and an anti-clumping agent added at various concentrations (X axis) to culture medium on the expansion of WA09 hESCs (top row, “H9-ESC”) and Gibco Human Episomal iPSCs (middle row, “Gepi”) grown in suspension culture, as described in EXAMPLE 2.

FIG. 2 depicts phase contrast micrographs [40×] of iPSCs grown in suspension culture under various conditions as described in EXAMPLE 2. In the left column of micrographs under the heading “Continuous”, cells were continuously exposed to the indicated small molecules at the indicated concentrations. In the right column of micrographs under the heading “Day 1,” the cells were exposed to the indicated small molecules at the indicated concentration on day 1 only.

FIG. 3 is a bar graph showing the fold-change assessment of impact of daily addition of various small molecules (blue bars, “Continuous”) vs. addition of the small molecules on Day 1 (red bars, “Day One”), relative to PSC base culture media (green bars) without anti-clump reagent (control) or with anti-clump reagent (Control+DS) to suspension cultures of iPSCs. The Y-axis indicates the fold-change in viable cell counts from day 1 to day 5 of culture. Representative micrographs of the cell cultures at day 5 under certain conditions are shown.

FIG. 4 is a bar graph showing the fold-change assessment of impact of daily addition of various small molecules (blue bars, “Continuous”) vs. addition of the small molecules on Day 1 (red bars, “Day One”), relative to base culture media (green bars) without anti-clump reagent (control) or with anti-clump reagent (Control+DS) to suspension cultures of WA09 ESCs. The Y-axis indicates the fold-change in viable cell counts from day 1 to day 5 of culture. Representative micrographs of the cell cultures at day 5 under certain conditions are shown.

FIGS. 5A-5B show assessment of impact graphs of the ROCK inhibitors Y-27632, Pinacidil, or RevitaCell on expansion of iPSC's grown in suspension culture in the presence of CHIR99021 (3 uM) or 10 uL anti-clump reagent as described in EXAMPLE 3.

FIG. 6 depicts phase contrast micrographs of cells grown in suspension culture in base culture media containing various concentrations of BSA in the presence or absence of CHIR99021 as indicated, as described in EXAMPLE 3.

FIG. 7 depicts phase contrast micrographs of iPSCs grown in suspension culture at in the presence of the indicated concentrations of CHIR99021 and BSA, at day 5 of culture, and immunocytochemical micrographs of the same expanded cells upon replating stained for OCT4 (green) and DNA (blue), as described in EXAMPLE 3.

FIG. 8 shows assessment of impact graphs of the addition of various concentrations of Pinacidil, CHIR99021, Go6893 and BSA (as indicated) to base PSC culture medium on the expansion of iPSCs grown in suspension culture, as described in EXAMPLE 3.

FIGS. 9A-9E show PSC expansion in suspension culture and maintenance of pluripotency using ROCKi, PSC media formulations and a commercially available media. for PSC suspension cultures. The graphs of FIGS. 9A-9B depict expansion fold-change of (A) Gibco Human Episomal iPSCs and (B) WA09 (H9) ESCs in the indicated media formulations The graph of FIG. 9C depicts maintenance of pluripotency with the indicated media formulations (x-axis) as assessed via % OCT4 positive cells (iPSCs (blue bars); ESCs (red bars)). FIG. 9D shows a phase contrast micrograph of ESCs spontaneously differentiated following expansion and FIG. 9E depicts trilineage differentiation of the expanded ESCs assessed via the TaqMan™ hPSC Scorecard™ Panel.

FIGS. 10A-10C show ROCKi pairing with PSC culture media formulations for PSC expansion. FIG. 10A depicts fold-change in iPSC expansion with the pairing of various concentrations of Pinacidil, Thiazovivin or Y-27632 with base PSC culture medium excluding CHIR99021 as described in EXAMPLE 3. FIG. 10B depicts phase contrast micrographs of iPSCs seeded with Y-27632 or Pinacidil and cultured in PSC media with or without CHIR99021. FIG. 10C is a graph which depicts three passage cumulative fold-change in expansion of iPSCs cultured with the following PSC media formulation: 1) PSC medium+1× RevitaCell, 2) PSC medium+1× RevitaCell+1.88 μM CHIR99021, 3) PSC medium+10 μM Y-27632, 4) PSC medium+10 μM Y-27632+1.88 μM CHIR99021.

FIG. 11A-B depicts fold-change expansion (A) and viability (B) of iPSCs expanded in 100 mL bioreactors with base PSC media with various sugar sources and concentrations of CHIR99021 relative to a commercially available medium, mTeSR1. Media exchange was completed in an every day (ED) or every-other-day (EOD) cadence as described in Example 3.

FIGS. 12A-12D show an assessment of ROCKi pairing with PSC culture medium formulation. FIG. 12A is a graph of iPSC expansion at day 5 when seeded with RevitaCell and with Y-27632. FIG. 12B is a graph of a 3 passage expansion of iPSCs when seeded with Pinacidil and with Y-27632. FIG. 12C is a graph of a 3 passage expansion of iPSCs when seeded with Chroman I and with Y-27632. FIG. 12D are phase contrast micrographs of iPSC spheroids generated using the indicated ROCKi with the PSC medium formulation.

FIG. 13 is a graph which depicts fold-expansion of PSCs in suspension culture grown using media formulations: (A) PSC media in the absence of CHIR99021, (B) PSC media containing CHIR99021, and (C) mTeSR1.

FIGS. 14A-C show a comparison of iPSC expansion and pluripotency from the use of complete PSC media formulation and the use of mTeSR1 media. The graph of FIG. 14A depicts iPSC expansion fold-change over 3 passages. The graph of FIG. 14B depicts viability over the course of passaging. The graph of FIG. 14C depicts maintenance of pluripotency as assessed via intracellular markers of pluripotency OCT4 and NANOG.

FIGS. 15A-15B show assessment of spheroid growth using RevitaCell, Y-27632 and Cellartis Supplement 3 for seeding. FIG. 15A depicts expansion fold-change and phase contrast micrographs of spheroids from the noted seeding conditions. FIG. 15B depicts pluripotency of the spheroids from the noted seeding conditions.

FIG. 16 is a graph depicting the ability of PSCs expanded in complete PSC medium and two other expansion media to then be directly differentiated to the endodermal lineage.

FIG. 17A-17B depict ICC images and graphs showing the ability of cells expanded in complete PSC medium formulation to then be directly differentiated to ectodermal lineage. FIG. 17A depicts representative ICC images showing expression of SOX1 (red) and Nestin (green) with quantitation of SOX1 expression shown in the graph below. FIG. 17B depicts representative ICC images showing expression of PAX6 (red) and OTx2 (green) with quantitation of PAX6 shown in the graph below.

FIGS. 18A-18D shows PSC expansion and pluripotency in a 100 mL format using a PBS bioreactor. The graph of FIG. 18A depicts fold-expansion iPSCs over 3 passages. FIGS. 18B-18C depicts maintenance of pluripotency as determined via TaqMan™ hPSC Scorecard™ Panel (B) and PluriTest™ Assay analysis (C). FIG. 18D shows maintenance of normal karyotype assessed via KaryoStat Assay analysis.

FIGS. 19A-19C show the assessment of spheroid diameter tuning in PBS minibioreactors at 100 mL scale. The image in FIG. 19A indicate the spheroid morphology on Day 3 post-initiation indicating the ability to generate spheroids of various diameters. The graph of FIG. 19B shows fold-change at Day 5 post-passage for bioreactor cultures with various stir speeds (x-axis). Cell counts from each individual flask is indicated by an individual data point. The graph of FIG. 19C depicts pluripotency for bioreactor cultures with various stir speeds (x-axis) via the presence of extracellular (TRA1-60, blue) and intracellular (OCT4, red) markers of pluripotency.

FIGS. 20A-20D show spheroid growth from different cell lines in shaker flasks and other vessels. FIG. 20A depicts representative phase contrast micrographs of spheroids and FIG. 20B depicts cumulative fold-expansion from Gibco Episomal iPSCs, WTC-11 iPSCs, WA09 ESCs, and WA01 ESCs grown in shaker flasks. FIGS. 20C-20D depict fold-expansion of iPSCs in various size shaker flasks (C) and multiple types of culture vessels (D).

### DETAILED DESCRIPTION

Described herein are cell culture compositions that comprise molecule GSK3 inhibitor, a ROCK inhibitor, one or more mitogenic growth factors. Advantageously, the cell culture compositions described herein increase pluripotent stem cell (PSC) growth, enhance PSC proliferation, maintain PSC pluripotency, maintain spheroid morphology, maintain PSC morphology, increase PSC passage count, increase PSC culture scale, as compared to customary PSC media.

As used herein “stem cells” refer to undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts. Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multipotent stem cells; or (5) unipotent, meaning able to give rise to a single cell lineage (e.g., spermatogenic stem cells).

The skilled artisan readily appreciates that pluripotent stem cells may express one or more “pluripotency markers,” or biomolecules indicative of pluripotent cells, including but not limited to SSEA 3 and 4, alkaline phosphatase, nanog, Oct4, Sox2 and the like. Pluripotency markers can be measured using art-accepted techniques, including but not limited to antibody-based assays, PCR-based assays, hybridization based assays, cytochemistry-based assays, histochemistry-based assays and the like. Several commercially available tests are available.

Propagated pluripotent stem cells have potential to differentiate into cells of all three germinal layers: endoderm, mesoderm, and ectoderm tissues. Pluripotency of stem cells can be confirmed, for example, by injecting cells into severe combined immunodeficient (SCID) mice, fixing the teratomas that form using 4% paraformaldehyde, and then examining them histologically for evidence of cell types from the three germ layers. Alternatively, pluripotency may be determined by the creation of embryoid bodies and assessing the embryoid bodies for the presence of markers associated with the three germinal layers. Propagated pluripotent stem cell lines may be karyotyped using a standard G-banding technique and compared to published karyotypes of the corresponding primate species. Cells may have a “normal karyotype,” which means that the cells are euploid, wherein all human chromosomes are present and not noticeably altered.

Pluripotent stem cells useful in the embodiments described herein include established lines of pluripotent cells derived from tissue formed after gestation, including pre-embryonic tissue (such as, for example, a blastocyst), embryonic tissue, or fetal tissue taken any time during gestation, typically but not necessarily before approximately 10-12 weeks gestation. Non-limiting examples include established lines of human embryonic stem cells or human embryonic germ cells, such as, for example the human embryonic stem cell lines H1, H7, and H9. Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant human embryonic stem cell lines, such as, for example, BG01v. Also suitable are pluripotent stem cells derived from non-pluripotent cells, such as, for example, adult somatic cells.

As used herein, the phrase “induced pluripotent stem (iPS) cell (iPSC)” (or embryonic-like stem cell) as used herein refers to a proliferative and pluripotent stem cell which is obtained by de-differentiation of a somatic cell (e.g., an adult somatic cell).

As used herein “differentiation” refers to the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, “de-differentiation” refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, “maintenance” refers generally to cells placed in a growth medium under conditions that facilitate cell growth, expansion, and/or division that may or may not result in a larger population of the cells.

As used herein, “passaging” refers to the process of removing cells from one culture vessel and placing them in a second culture vessel under conditions that facilitate cell growth/expansion and/or division. In some embodiments, passaging can include dissociation of cell clusters to obtain smaller clusters or individual cells, followed by growth of the dissociated clusters or cells in culture media. In some embodiments, all or a portion of the dissociated cell clusters or cells are placed in new culture media. In some embodiments, the dissociated clusters or cells are not placed in new media, but rather, additional media or supplements are added to the dissociated cell culture. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore, the number of population doublings of a culture is greater than the passage number. The expansion of cells (i.e., the number of population doublings) during the period between passaging depends on many factors, including but not limited to the seeding density, substrate, medium, growth conditions, and time between passaging.

In some embodiments, the PSC seeding density for suspension culture is about 25,000 to about 400,000 viable cells/mL. In some embodiments, the PSC seeding density for suspension culture is about 100,000 to about 200,000 viable cells/mL. In certain embodiments, the PSC seeding density is about 150,000 viable cells/mL. In some embodiments, the PSC seeding density is about 25,000, about 50,000, about 75,000, about 100,000, about 125,000, about 175,000, about 200,000, about 225,000, about 250,000, about 275,000, about 300,000, about 325,000, about 350,000, about 375,000, about 400,000, about 25,000 to about 100,000, about 50,000 to about 200,000, about 100,000 to about 300,000, or about 200,000 to about 400,000 viable cells/mL.

As used herein the term “expanding” as used in the context of expanding PSC cultures, refers to the viable cells achieved over the culturing period/viable cells seeded in the culture. As such, expanding can refer to the fold-increase and/or percent increase of the viable PSC count over the culturing period. It will be appreciated that the number of pluripotent stem cells, which can be obtained from a single pluripotent stem cell, depends on the proliferation capacity of the pluripotent stem cell. The proliferation capacity of a pluripotent stem cell can be calculated by the doubling time of the cell (i.e., the time needed for a cell to undergo a mitotic division in the culture) and the period the pluripotent stem cell culture can be maintained in the undifferentiated state (which is equivalent to the number of passages multiplied by the days between each passage).

As used herein the phrase “dissociation” in the context of dissociating PSCs refers to separating PSC clumps or clusters to smaller clumps or clusters and/or single cells. Mechanical and non-mechanical dissociation means are useful in the embodiments described herein.

As used herein the phrase “single cells” refers to the state in which the pluripotent stem cells do not form cell clusters. In some embodiments, “single cells” refers to less than or equal to 10 PSCs clustered together, e.g., 1, 2, 3, 4, 5, 6, 7, 8, 9, or 10 cells. In some embodiments, a PSC “cluster” refers to the aggregation of about 200 or more pluripotent stem cells, in the suspension culture. In some embodiments, each of the pluripotent stem cell clumps comprises at least about 200 cells, at least about 500 cells, at least about 600 cells, at least about 700 cells, at least about 800 cells, at least about 900 cells, at least about 1000 cells, at least about 1100 cells, at least about 1200 cells, at least about 1300 cells, at least about 1400 cells, at least about 1500 cells, at least about 5×10³ cells, at least about 1×10⁴ cells, at least about 5×10⁴ cells, at least about 1×10⁵ cells, at least about 1×10⁶ cells or more.

As used herein “suspension culture” refers to a culture in which the pluripotent stem cells are suspended in a medium rather than adhering to a surface.

As used herein “markers” refer to biomolecules (e.g., nucleic acids, proteins, glycoproteins, etc.) that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art. Markers of pluripotency include, but are not limited to, SSEA3, SSEA4, TRA-1-60, TRA-1-81, CD24, OCT4, NANOG, and alkaline phosphatase (AP).

As used herein “customary PSC media” or “customary PSC medium” refer to any PSC media known in the art such as MEF (mouse embryonic fibroblast)-conditioned media or feeder-free systems such as StemFlex™ medium, Nutristem® hESC XF culture medium, Essential 8™ media, mTeSR™ media, mTeSR™-2 media, etc.

As used herein the term “purine derivative” refers to the purine heterocyclic compound and variations thereof. In another aspect, purine derivative refers to a purine nucleoside, a purine nucleotide, or a purine nucleotide mono-, di-, or tri-phosphate. In one aspect, “purine derivative” refers to purine, adenine, allopurinol, caffeine, dyphylline, guanine, hypoxanthine, isoguanine, theobromine, theophylline, uric acid, xanthine, inter alia, salts thereof, esters thereof, or combinations thereof.

As used herein, the phrases “medium or supplement concentrate,” “concentrated medium, supplement, or medium,” “concentrate” or “#x concentrate” are used interchangeably and refer a solution containing one or more species at a concentration greater than the intended concentration for use, i.e., the “working concentration.” In the case of “#x concentrate,” the “#x” refers to the dilution factor. For example, a “10× concentrate” would be diluted 10-fold to achieve a 1× working concentration. A 10× concentrate (e.g., 2 M) is diluted by combining 1 part of the 10× concentration with 9 parts of a solvent, such as water, to achieve a 1× working concentration (e.g., 200 mM). The dilution equation, C1·V1=C2·V2, where C1 is the initial concentration, V1 is the initial volume, C2 is the final concentration, and V2 is the final volume, can be used to calculate the appropriate dilution.

As used herein, the terms “powder” or “dry powder” refer to feed, supplement, or media powders or powdered media compositions for cell culture that are present in dry granular form, whose gross appearance may be free flowing. The term “powder” includes agglomerated powders. Preparation of agglomerated media, feeds, nutritive powders, supplements, etc.; their properties; and methods to prepare auto pH and auto osmolarity of agglomerated media, feeds, nutritive powders, supplements, etc. have been described in U.S. Pat. Nos. 6,383,810 and 6,627,426 and U.S. Pat. App. Pub. No. US 2019/0048312 A1, inter alia, each of which is incorporated by reference for teachings related to agglomerated media.

As used herein, the term “ingredient” refers to any compound, whether of chemical or biological origin, that can be used in cell culture media to maintain or promote the growth of proliferation of cells. The terms “component,” “nutrient,” and ingredient” are used interchangeably and all refer to such compounds. Typical ingredients that are used in cell culture media include amino acids, salts, metals, sugars, carbohydrates, lipids, nucleic acids, hormones, vitamins, fatty acids, proteins, and the like. Other ingredients that promote or maintain cultivation of cells ex vivo can be selected by those of skill in the art, in accordance with the particular need.

As used herein, the terms “cell culture” or “culture” refer to the maintenance of cells in an artificial, e.g., an in vitro environment. It is to be understood, however, that the term “cell culture” is a generic term and may be used to encompass the cultivation not only of cells such as human or animal cells, individual prokaryotic (e.g., bacterial) or eukaryotic (e.g., animal, plant and fungal) cells, but also of tissues, human or animal tissues, organs, organ systems or whole organisms, for which the terms “tissue culture,” “organ culture,” “organ system culture” or “organotypic culture” may be used interchangeably with the term “cell culture.”

As used herein, the phrases “cell culture medium,” “culture medium,” “medium formulation,” “pluripotent stem cell composition,” “PSC composition”, “PSC medium”, or “medium” (plural “media” in each case) refer to a nutritive solution or “nutritive medium” that supports the cultivation and/or growth of cells; these phrases may be used interchangeably. A cell culture medium may be a basal medium (a general medium that requires additional ingredients to support cell growth) or a complete medium that has all or almost all components to support cell growth. Cell culture media may be serum-free, protein-free (one or both), animal origin free, may or may not require additional components like small molecules, growth factors, additives, feeds, supplements, for efficient and robust cell performance.

As used herein “base media,” or “basal media” refers to a medium that typically requires supplementation to support cell growth. Basal medium may include vitamins and amino acids, but it does not include proteins, lipids, small molecules, or growth factors.

Nutritive media and supplements as described herein can be divided into various “subgroups” that can be prepared and used as described herein. Such subgroups can be prepared separately and then combined to produce a nutritive medium. For examples of compatible subgroups and related considerations see U.S. Pat. Nos. 5,474,931 and 5,681,748, which are incorporated by reference herein for such teachings.

As used herein, the term “combining” refers to the mixing or admixing of ingredients in a cell culture medium formulation. Combining can occur in liquid or powder form or with one or more powders and one or more liquids. In one example, two or more components may be mixed to produce a complex mixture such as media or supplements, media subgroups, or buffers. Combining also includes mixing dry components with liquid components.

As used herein, the term “contacting” refers to the placing of cells to be cultivated into a culture vessel with the medium and/or supplement in which the cells are to be cultivated. The term “contacting” encompasses inter alia mixing cells with medium and/or supplement, perfusing cells with medium and/or supplement, pipetting medium and/or supplement onto cells in a culture vessel, and submerging cells in culture medium and/or supplement.

As used herein, the term “cultivation” refers the maintenance of cells in an artificial environment under conditions favoring growth, differentiation, or continued viability, in an active or quiescent state, of the cells. Thus, “cultivation” may be used interchangeably with “culturing,” “cell culture,” “growing cells,” “maintaining cells,” or any of the synonyms described above.

As used herein, the term “culture vessel” refers to a receptacle for holding cells. The vessel may be glass, plastic, metal, or other material that can provide an aseptic environment for culturing, holding, or storing cells. The culture vessel may be a plate with wells, such as a 6-well plate, 12-well plate, 24-well plate, or 96-well plate. The plate may be a non-tissue culture treated plate that can be used for a variety of cell culture applications. The plates may have a clear, untreated, hydrophobic polystyrene surface that is sterile. The plate may have a lid that is non-reversible and prevents cross-condensation among wells. The culture vessel may be a shaker flask, spinner flask, bioreactor, suspension bag, or other means for culturing cells. In some embodiments, the culture vessel may be a 125 mL shaker flask, a 250 mL shaker flask, a 100 mL bioreactor, or a 500 mL bioreactor. The term “container” is synonymous.

In some embodiments, PSCs are grown in a suspension culture volume of about 1 mL to about 1000 mL. In some embodiments, PSCs are grown in a suspension culture volume of about 0.5 mL to about 2 mL, 0.5 mL to about 20 mL, about 20 mL to about 100 mL, about 20 mL to about 500 mL, about 100 mL to about 500 mL, or about 500 mL to about 1000 mL.

In some embodiments, culturing or growing PSCs includes agitating the cells during the cell culture. In some embodiments, PSCs in suspension culture are subjected to agitation or an agitating motion for most of the culture period. In some embodiments, PSCs in suspension culture are subjected to constant or continuous agitation or an agitating motion for most of the culture period. Exemplary means for agitating culture cells include without limitation rocker platforms, such as orbital rocker platforms; stir plates, such as magnetic stir plates; orbital shakers, such as CO2 resistant shakers; and bioreactors, for example a bioreactor with a rotating wheel impeller or a bioreactor with a stirring impeller. As described herein, the speed at which the PSC suspension culture is agitated may vary according to, without limitation, the volume of the suspension culture, the size and type of culture vessel, the means for agitating the culture, the cell density, and the spheroid size desired during culture. In some embodiments, culturing PSCs in suspension includes agitating the cells at least 20 RPM to about 200 RPM. In some embodiments, culturing PSCs in suspension includes agitating the cells at about 30 RPM to about 180 RPM, about 40 RPM to about 160 RPM or about 40 RPM to about 80 RPM. In certain embodiments, PSCs in suspension culture are agitated at a single speed throughout the culture period. In certain embodiments, PSCs in suspension culture are agitated at at least 2 different speeds during the culture period.

As used herein, the term “effective amount” or “effective concentration” refers to an amount of an ingredient, which is available for use. One example is the amount of a vitamin in a culture medium, which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one having ordinary skill in the art or by experimental determination.

As used herein, the terms “supplement,” “supplement composition,” “stem cell culture supplement composition,” “feed,” or “feed or supplement” refer to a composition when added to cells in standard culture may be beneficial for cell maintenance, expansion, growth, and viability, or may affect cell performance, may increase culture longevity, may increase cell proliferation, may maintain pluripotency, may maintain spheroid morphology, may maintains PSC morphology, may increase passage count, may increase culture scale or the like. The terms “feed” or “supplement” may be used interchangeably in this disclosure and refers to dry powders or liquid formats of media, feeds, or supplements comprising one or more small molecule inhibitors (such as a ROCK inhibitor and/or GSK3 inhibitor, or the like), amino acids, sugars, vitamins, inorganic or organic salts, trace elements, buffers, peptides, hydrolysates, fractions, growth factors (including mitogenic growth factors), albumins, hormones, etc. required to rebalance or replenish or to modulate the growth or performance of a cell in culture, or a cell culture system. A feed or supplement may be distinguished from a cell culture medium in that it is typically added to a cell culture medium that can be or is being used culture a cell (e.g., is added to an existing cell culture in a particular media or is added to a particular media before adding cells to the media). As cells grow in the media, components are consumed, and the feed or supplement replaces those depleted or degraded components. A portion of the media, supplement, or media comprising the supplement can be removed from a cell culture as cells grow in the media and replaced with an equivalent amount (i.e., exchanged) of fresh media, supplement, or media comprising the supplement. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition is exchanged. In some embodiments, at least 50% of the medium and supplement composition is exchanged. In other embodiments, a feed, supplement or medium containing a supplement may be added on top of a culture solution as an overlay. In addition, the feed or supplement can be used to modulate the cells within the culture by, for example, increasing cell viability, increasing cell proliferation, enhancing cell growth, maintaining pluripotency, maintaining morphology, or increasing passage count. As would be understood by one of ordinary skill in the art, a feed or supplement may comprise amino acids, sugars, vitamins, buffers, etc., required to rebalance or replenish or modulate the growth or performance of a cell in culture, or a cell culture system.

In one embodiment described herein, the feed or supplement includes at least one small molecule inhibitor, mitogenic growth factor, or albumin, salts, or esters thereof, or combinations thereof in a cell culture compatible vehicle. Such supplement can be added to a new or existing cell culture from time to time to increase cell growth, increase cell viability, increase culture longevity, increase cell proliferation, maintain pluripotency, maintain spheroid morphology, maintain PSC morphology, increase passage count, or increase culture scale. Such feed or supplement may be a concentrate or at a working concentration or may be partially concentrated for certain components only to account for dilution and maintain the concentrations of components present in the original media.

As used herein a cell culture medium composition is composed of a number of ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L l-arginine, 0.05 g/L l-asparagine, and 0.02 g/L l-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations and ingredients of a 1× formulation of cell culture medium are known to those of ordinary skill in the art. See, for example, Banes et al., Methods for Preparation of Media, Supplements and Substrate for Serum-Free Animal Cell Culture, Alan R. Liss, N.Y. (1984), which is incorporated by reference herein in its entirety. The osmolality and/or pH, however, may differ in a 1× formulation compared to the culture medium, particularly when fewer ingredients are contained in the 1× formulation. The 1× concentration of any component is not necessarily constant across various media formulations. 1× might therefore indicate different concentrations of a single component when referring to different media. However, when used generally, 1× will indicate a typical working concentration commonly found in the types of media being referenced. A 1× amount is the amount of an ingredient that will result in a 1× concentration for the relevant volume of medium.

As used herein, a “10× formulation” refers to a solution wherein each ingredient in that solution is about 10-times more concentrated than the same ingredient in the cell culture medium. For example, a 10× formulation of RPMI-1640 culture medium may contain, among other ingredients, 2.0 g/L l-arginine, 0.5 g/L l-asparagine, and 0.2 g/L l-aspartic acid (compare 1× formulation, above). A “10× formulation” may contain a number of additional ingredients at a concentration about 10 times that found in the 1× culture medium. As will be readily apparent, “20× formulation,” “25× formulation,” “50× formulation” and “100× formulation” designate solutions that contain ingredients at about 20-, 25-, 50- or 100-fold concentrations, respectively, as compared to a 1× cell culture medium. Again, the osmolality and pH of the media formulation and concentrated solution may vary. See U.S. Pat. No. 5,474,931, which is directed to culture media concentrate technology.

As used herein, “physiologic pH” is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc. or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. Some supplements, though not preferred, may only be entirely soluble outside these ranges.

As used herein, the phrase “without significant loss of biological and biochemical activity” refers to a decrease of less than about 30%, less than about 25%, less than about 20%, less than about 15%, or less than about 10%, of the biological or biochemical activity of the nutritive media, media supplement, media subgroup, buffer or sample of interest when compared to a freshly made nutritive media, media supplement, media subgroup, buffer or sample of the same formulation.

As used herein, a “solvent” is a liquid that dissolves or has dissolved another ingredient of the medium. Solvents may be used in preparing media, feeds, supplements, subgroups, or other formulations, and in reconstituting a medium or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five, six, or more components may be necessary in some cases to form a soluble mixture. Simple solvents such as mixtures of ethanol or methanol and water may be used because of their ease of preparation and handling.

As used herein, the term “extended period of time” or “long-term shelf life” interchangeably refer to a period of time longer than that for which the sample (e.g., pharmaceutical composition, nutritive medium, medium supplement, medium subgroup, or buffer) is stored. As used herein, an “extended period of time” or “long-term shelf life” therefore means about 1-36 months, about 2-30 months, about 3-24 months, about 6-24 months, about 9-18 months, or about 4-12 months, under a given storage condition, which may include storage at temperatures of about −70° C., about −20° C., about 0° C., about 4° C., about 10° C., about 20° C., about 25° C., about −70° C. to about 25° C., about −20° C. to about 25° C., about 0° C. to about 25° C., about 4° C. to about 25° C., about 10° C. to about 25° C., or about 20° C. to about 25° C. Assays for determining the biological or biochemical activity of pharmaceutical or clinical compositions, cell culture reagents, nutrients, nutritive media, media supplement, media subgroup, or buffers are well known in the art and are familiar to one of ordinary skill.

As used herein, the term “about” means “approximately” and when modifying a numerical value indicates that the value can vary by ±10% of the stated value.

One embodiment described herein is a pluripotent stem cell composition comprising a cell culture basal medium, at least one GSK3 inhibitor, one or more mitogenic growth factors, and optionally at least one ROCK inhibitor that when added to a cell culture, provides one or more of enhancing PSC growth, enhancing PSC proliferation, maintaining PSC pluripotency, maintaining spheroid morphology, maintaining PSC morphology, increasing PSC passage count, or increasing PSC culture scale as compared to customary PSC media. In some embodiments, the composition further comprises one or more albumins or peptides thereof. The composition described herein may also optionally contain one or more amino acids, sugars, vitamins, peptides, inter alia required to rebalance or replenish media components of a cell in culture or a cell culture system. In one aspect, the composition is a liquid concentrate that is diluted prior to or upon use in the cell culture. In another aspect, the feed is a dry powder, agglomerated powder, tablet, or other dry form that is added directly to the culture or is dissolved in a cell culture compatible vehicle (e.g., water) to achieve a concentrate or working volume prior to addition to a cell culture. In one aspect, the composition comprises components variously selected from the exemplary list of Table 1.

TABLE 1
Exemplary PSC Basal Medium and/or Supplement Components

| | | Stock | Working |
|---|---|---|---|
| | | Conc. | Conc. |
| Component | Exemplary Species | (10×) | (1×) |
| Lipids, fatty | Cholesterol, lipoic acid, caprylic acid, | 10× | Var* |
| acids | capric acid, lauric acid, myristic acid, | | |
| | palmitic acid,s tearic acid, | | |
| | arachidonic acid, linoleic acid, | | |
| | linolenic acid, oleic acid, palmitoleic | | |
| | acid, cholesterol synthetic, D/L- | | |
| | tocopherol acetate, behenic acid, | | |
| | lignoceric aid, cerotic acid, | | |
| | myristoleic acid, sapienic acid, elaidic | | |
| | acid, vaccenic acid, α-linolenic acid, | | |
| | erucic acid, eicosapentaenoic acid, | | |
| | docosahexaenoic acid | | |
| Amino acids, | Glycine, alanine, arginine, | 10× | Var* |
| salts, esters, | asparagine, aspartic acid, cysteine, | | |
| or di- or | cystine, glutamic acid, glutamine, | | |
| tri-peptides | histidine, isoleucine, leucine, lysine, | | |
| thereof | methionine, phenylalanine, proline, | | |
| | serine, threonine, tryptophan, | | |
| | tyrosine, valine | | |
| Inorganic | AgNO₃, AlCl₃, Ba(C₂H₃O₂)₂, CaCl₂, | 10× | Var* |
| salts, organic | CdSO₄, CdCl₂, CoCl₂, Cr₂(SO₄)₃, | | |
| salts | CuCl₂, CuSO₄, FeSO₄, FeCl₂, FeCl₃, | | |
| | Fe(NO₃)₃, GeO₂, Na₂SeO₃, H₂SeO₃, | | |
| | KBr, KCl, KI, MgCl₂, MgSO₄, | | |
| | MnCl₂, NaCl, NaF, Na₂SiO₃, NaVO₃, | | |
| | Na₃VO₄, (NH₄)₆Mo₇O₂₄, Na₂HPO₄, | | |
| | NaH₂PO₄, NaHCO₃, NiSO₄, NiCl₂, | | |
| | Ni(NO₃)₂, RbCl, SnCl₂, ZnCl₂, | | |
| | ZnSO₄, ZrOCl₂, pyridoxine | | |
| | hydrochloride (C₈H₁₂ClNO₃), sodium, | | |
| | potassium, magnesium, calcium, | | |
| | ammonium, phosphate, carbonate, | | |
| | bicarbonate, sulfate, citrate, acetate, | | |
| | or nitrate | | |
| Proteins, | Insulin, transferrin, epidermal growth | 10× | Var* |
| peptides, | factor (EGF), transforming growth | | |
| mitogenic | factor alpha (TGF-α), transforming | | |
| growth | growth factor beta (TGF-β), basic | | |
| factors, | fibroblast growth factor (bFGF), | | |
| albumins, | brain-derived neurotrophic factor | | |
| recombinant | (BDNF), hepatocyte growth factor | | |
| extracellular | (HGF), heregulin (HRG), | | |
| matrix | keratinocyte growth factor (KGF), | | |
| proteins, salts, | Activin A, vitronectin (VTN-N), | | |
| esters, or di- | bovine serum albumin (BSA), human | | |
| or tri-peptides | seruma lbumin (HSA), recombinant | | |
| thereof | human albumins, GlutaMAX-I, L- | | |
| | alanyl-L-glutamine, putrescine 2 HCl | | |
| Vitamins, | Biotin (B7), choline, folic acid (B9), | 10× | Var* |
| salts, or esters | niacinamide (B3), pyridoxine (B6), | | |
| thereof | riboflavin (B2), thiamine (B1), | | |
| | cobalamin (B12), inositol, retinol (A), | | |
| | pantothenic acid (B5), ascorbic acid | | |
| | (C), cholecalciferol (D), tocopherol | | |
| | (E), phylloquinone (K), lipoic acid, | | |
| | linoleic acid, para-aminobenzoic acid | | |
| Purine | Purine, adenine, guanine, xanthine, | 10× | Var* |
| derivatives, | thymidine, hypoxanthine, | | |
| salts, or esters | nucleosides, nucleotides | | |
| thereof | | | |
| Other | Water, buffering agents, sugars, | 10× | Var* |
| additives | detergents, solvents, ethyl alcohol, | | |
| | carbon source (carbohydrate or | | |
| | carboxylic acid derivatives), trace | | |
| | minerals, minerals, antioxidants, | | |
| | serum or serum replacements, pH | | |
| | indicators, antibiotics, antimycotics, | | |
| | thiols, surfactants, etc. | | |
| Inhibitors | CHIR99021 (6-[[2-[[4-(2,4- | — | 1 μM- |
| | dichlorophenyl)-5-(5-methyl-1H- | | 50 μM |
| | imidazol-2-yl)-2- | | |
| | pyrimidinyl]amino]ethyl]amino]-3- | | |
| | pyridinecarbonitrile), BIO ((2′Z,3′E)- | | |
| | 6-bromoindirubin-3′-oxime), AR-A | | |
| | 014418 (N-[(4- | | |
| | methoxyphenyl)methyl]-N′-(5-nitro- | | |
| | 2-thiazolyl)urea)0, Kenpaullone (9- | | |
| | bromo-7,12-dihydro-indolo[3,2- | | |
| | d][1]benzazepin-6(5H)-one), SB | | |
| | 216763 (dichlorophenyl)-4-(1- | | |
| | methyl-1H-indol-3-yl)-1H-pyrrole- | | |
| | 2,5-dione), SB 415286 (3-[(3-chloro- | | |
| | 4-hydroxyphenyl)amino]-4-(2- | | |
| | nitrophenyl)-1H-pyrrole-2,5-dione), | | |
| | Y-27623 ((R)-(+)-trans-4-(1- | | |
| | aminoethyl)-N-(4- | | |
| | pyridyl)cyclohexanecarboxamide), | | |
| | Pinacidil, RevitaCell, Chroman 1, | | |
| | Emricasan, Polyamines, Trans- | | |
| | ISRIB, thiazovavin, or CEPT | | |

*Var: Various concentrations; q.s. quantum sufficit.

In one embodiment, the pluripotent stem cell composition or supplement composition comprises at least one small molecule inhibitor, a salt thereof, an ester thereof, or a combination thereof. In one aspect, the small molecule inhibitor is a glycogen synthase kinase-3 (GSK3) inhibitor, salts thereof, or esters thereof, or a rho kinase (ROCK) inhibitor, salts thereof, or esters thereof. As used herein, the term “GSK3 inhibitor” includes the inhibitor molecule, salts thereof and esters thereof. As used herein, the term “ROCK inhibitor” or “ROCKi” includes the inhibitor molecule, salts thereof and esters thereof. In one aspect, pluripotent stem cell composition or supplement composition comprises a small molecule GSK3 inhibitor and a ROCK inhibitor. In another aspect, the GSK3 inhibitor comprises one or more of CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), BIO ((2′Z,3′E)-6-bromoindirubin-3′-oxime), AR-A 014418 (N-[(4-methoxyphenyl)methyl]-N′-(5-nitro-2-thiazolyl)urea)0, Kenpaullone (9-bromo-7,12-dihydro-indolo[3,2-d][1]benzazepin-6(5H)-one), SB 216763 (dichlorophenyl)-4-(1-methyl-1H-indol-3-yl)-1H-pyrrole-2,5-dione), or SB 415286 (3-[(3-chloro-4-hydroxyphenyl)amino]-4-(2-nitrophenyl)-1H-pyrrole-2,5-dione), inter alia, salts thereof, esters thereof, or combinations thereof. In another aspect, the ROCK inhibitor inhibits ROCK1 activity or ROCK2 activity. In another aspect, the ROCK inhibitor inhibits ROCK1 activity and ROCK2 activity. In another aspect, the rho kinase inhibitor comprises one or more of Y-27632 ((R)-(+)-trans-4-(1-aminoethyl)-N-(4-pyridyl)cyclohexanecarboxamide), Chroman 1, Emricasan, Polyamines, Trans-ISRIB, Pinacidil or thiazovavin, inter alia, salts thereof, esters thereof, or combinations thereof. A small molecule inhibitor combination of Chroman 1, Emricasan, Polyamines, and Trans-ISRIB (CEPT) has been described in Chen et al., BioRxiv (October 2019) doi.org/10.1101/815761, which is incorporated herein by reference for such teachings.

In another embodiment, the pluripotent stem cell composition or supplement composition comprises at least one mitogenic growth factor, a salt thereof, an ester thereof, or a combination thereof. In one aspect, the mitogenic growth factor is epidermal growth factor (EGF), transforming growth factor alpha (TGF-α), transforming growth factor beta (TGF-β), basic fibroblast growth factor (bFGF), Activin A, brain-derived neurotrophic factor (BDNF), hepatocyte growth factor (HGF), heregulin (HRG), keratinocyte growth factor (KGF), vascular endothelial growth factor (VEGF), platelet-derived growth factor (PDGF), insulin-like growth factor (IGF), multiplication-stimulating factor (MSF), sarcoma growth factor (SGF), nerve growth factor (NGF), fibroblast growth factor (FGF), granulocyte colony stimulating factor (G-CSF), granulocyte macrophage-colony stimulating factor (GM-CSF), Erythropoetin, thrombopoietin (TPO), bone morphogenic protein (BMP), growth differentiation factor (GDF), Neurotrophins, skeletal growth factor (SGF), inter alia, salts thereof, esters thereof, di- or tri-peptides, or combinations thereof.

In one aspect, the pluripotent stem cell composition or supplement composition comprises one or more mitogenic growth factors selected from Activin A, EGF, TGF-α, TGF-β, bFGF, BDNF, HGF, HRG, or KGF, inter alia, salts thereof, esters thereof, di- or tri-peptides, or combinations thereof. In another aspect, the pluripotent stem cell composition or supplement composition comprises two or more mitogenic growth factors selected from EGF, TGF-α, TGF-β, bFGF, BDNF, HGF, HRG, or KGF, inter alia, salts thereof, esters thereof, di- or tri-peptides, or combinations thereof. In another aspect, the pluripotent stem cell composition or supplement composition comprises at least two mitogenic growth factors selected from EGF and TGF-α, EGF and TGF-β, EGF and bFGF, EGF and BDNF, EGF and HGF, EGF and HRG, EGF and KGF, TGF-α and TGF-β, TGF-α and bFGF, TGF-α and BDNF, TGF-α and HGF, TGF-α and HRG, TGF-α and KGF, TGF-β and bFGF, TGF-β and BDNF, TGF-β and HGF, TGF-β and HRG, TGF-β and KGF, bFGF and BDNF, bFGF and HGF, bFGF and HRG, bFGF and KGF, BDNF and HGF, BDNF and HRG, BDNF and KGF, HGF and HRG, HGF and KGF, HRG and KGF, inter alia, salts thereof, esters thereof, or di- or tri-peptides thereof.

In one embodiment, the pluripotent stem cell composition or supplement composition comprises at least one albumin, peptides thereof, or combinations thereof. In one aspect, the albumin may be derived from human (HSA), bovine (BSA), fetal bovine (FBS), rat, mouse, horse, monkey, or pig sera. In one aspect, the albumin may be a recombinant albumin, including without limitation recombinant HSA or peptides thereof. In one aspect, the albumin is HSA, BSA, FBS, peptides thereof, or combinations thereof.

The concentrations of the small molecule inhibitors, mitogenic growth factors, and albumins in the pluripotent stem cell composition or supplement composition can be similar or different and may vary depending upon the application and cell type or culture type (e.g. plates, flasks, shake flasks, bioreactors, etc.). In addition, the pluripotent stem cell composition or supplement composition can be at working strength or a concentrate that is diluted prior to or during use. The pluripotent stem cell composition or supplement composition can be a powder or liquid that is added directly to the culture.

In one embodiment, the small molecule inhibitors, salt thereof, ester thereof, or combination thereof has a working concentration of about 0.5 μM to about 100 μM, including each integer within the specified range. In another embodiment, the mitogenic growth factor, salt thereof, ester thereof, di- or tri-peptides thereof, or combination thereof has a working concentration of about 0.1 ng/ml to about 1000 ng/ml, including each integer within the specified range. In another embodiment, the albumin, salt thereof, ester thereof, di- or tri-peptides thereof, or combination thereof has a working concentration of about 0.10% to about 3%, including each integer within the specified range.

In another embodiment, the small molecule inhibitor, salt thereof, ester thereof, or combination thereof has a 10× concentrate concentration of about 5 μM to about 1 mM, including each integer within the specified range. In another embodiment, the mitogenic growth factor, salt thereof, ester thereof, di- or tri-peptides thereof, or combination thereof has a 10× concentrate concentration of about 1.0 ng/ml to about 10,000 ng/ml, including each integer within the specified range. In another embodiment, the albumin, salt thereof, ester thereof, di- or tri-peptides thereof, or combination thereof has a 10× concentrate concentration of about 1% to about 30%, including each integer within the specified range.

In one embodiment, the small molecule inhibitor, salt thereof, ester thereof, or combination thereof has a concentration of about: 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM, 1.1 μM, 1.15 μM, 1.2 μM, 1.25 μM, 1.3 μM, 1.35 μM, 1.4 μM, 1.45 μM, 1.5 μM, 1.55 μM, 1.6 μM, 1.65 μM, 1.7 μM, 1.75 μM, 1.8 μM, 1.85 μM, 1.9 μM, 1.95 μM, 2 μM, 2.5 μM, 3 μM, 3.5 μM, 4 μM, 4.5 μM, 5 μM, 10 μM, 20 μM, 30 μM, 40 μM, 50 μM, 60 μM, 70 μM, 80 μM, 90 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM, 900 μM, 910 μM, 920 μM, 930 μM, 940 μM, 950 μM, 960 μM, 970 μM, 980 μM, 990 μM, 1000 μM, 0.1 mM, 0.2 mM, 0.5 mM, 0.75 mM, 1 mM, 2.5 mM, 5 mM, 7.5 mM, 10 mM, 15 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, or about 500 mM.

In one embodiment, the small molecule inhibitor, salt thereof, ester thereof, or combination thereof has a concentration of about 0.1 μM to about 1 μM, about 0.2 μM to about 1 μM, about 0.3 μM to about 1 μM, about 0.4 μM to about 1 μM, about 0.5 μM to about 1 μM, about 0.6 μM to about 1 μM, about 1 μM to about 5 μM, about 1 μM to about 4 μM, about 1 μM to about 3 μM, about 1 μM to about 2 μM, about 1 μM to about 10 μM, about 1 μM to about 20 μM, about 1 μM to about 50 μM, about 1 μM to about 100 μM, about 1 μM to about 200 μM, about 1 μM to about 500 μM, about 1 μM to about 1000 μM; about 10 μM to about 20 μM, about 10 μM to about 30 μM, about 10 μM to about 40 μM, about 10 μM to about 40 μM, about 10 μM to about 60 μM, about 10 μM to about 70 μM, about 10 μM to about 80 μM, about 10 μM to about 90 μM, about 10 μM to about 100 μM; about 100 μM to about 200 μM, about 100 μM to about 300 μM, about 100 μM to about 400 μM, about 100 μM to about 500 μM, about 100 μM to about 600 μM, about 100 μM to about 700 μM, about 100 μM to about 800 μM, about 100 μM to about 900 μM, about 100 μM to about 1000 μM; about 1 mM to about 5 mM, about 1 mM to about 10 mM, about 1 mM to about 20 mM, about 1 mM to about 50 mM, about 1 mM to about 100 mM, about 1 mM to about 200 mM, about 1 mM to about 500 mM, about 1 mM to about 1000 mM; about 10 mM to about 20 mM, about 10 mM to about 30 mM, about 10 mM to about 40 mM, about 10 mM to about 40 mM, about 10 mM to about 60 mM, about 10 mM to about 70 mM, about 10 mM to about 80 mM, about 10 mM to about 90 mM, about 10 mM to about 100 mM; about 100 mM to about 200 mM, about 100 mM to about 300 mM, about 100 mM to about 400 mM, about 100 mM to about 500 mM, about 100 mM to about 600 mM, about 100 mM to about 700 mM, about 100 mM to about 800 mM, about 100 mM to about 900 mM, or about 100 mM to about 1000 mM.

In one embodiment, the mitogenic growth factor, salt thereof, ester thereof, di- or tri-peptide thereof, or combination thereof has a working concentration of about 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml, 1.0 ng/ml, 2.0 ng/ml, 3.0 ng/ml, 4.0 ng/ml, 5.0 ng/ml, 6.0 ng/ml, 7.0 ng/ml, 8.0 ng/ml, 9.0 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, 100 ng/ml, 110 ng/ml, 120 ng/ml, 140 ng/ml, 160 ng/ml, 180 ng/ml, 200 ng/ml, 225 ng/ml, 250 ng/ml, 275 ng/ml, 300 ng/ml, 325 ng/ml, 350 ng/ml, 375 ng/ml, 400 ng/ml, 425 ng/ml, 450 ng/ml, 475 ng/ml, 500 ng/ml, 525 ng/ml, 550 ng/ml, 575 ng/ml, 600 ng/ml, 625 ng/ml, 650 ng/ml, 675 ng/ml, 700 ng/ml, 725 ng/ml, 750 ng/ml, 775 ng/ml, 800 ng/ml, 825 ng/ml, 850 ng/ml, 875 ng/ml, 900 ng/ml, 925 ng/ml, 950 ng/ml, 975 ng/ml, or about 1000 ng/ml.

In one embodiment, the mitogenic growth factor, salt thereof, ester thereof, di- or tri-peptide thereof, or combination thereof has a 10× concentrated concentration of about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1000 ng/ml, 1100 ng/ml, 1200 ng/ml, 1400 ng/ml, 1600 ng/ml, 1800 ng/ml, 2000 ng/ml, 2250 ng/ml, 2500 ng/ml, 2750 ng/ml, 3000 ng/ml, 3250 ng/ml, 3500 ng/ml, 3750 ng/ml, 4000 ng/ml, 4250 ng/ml, 4500 ng/ml, 4750 ng/ml, 5000 ng/ml, 5250 ng/ml, 5500 ng/ml, 5750 ng/ml, 6000 ng/ml, 6250 ng/ml, 6500 ng/ml, 6750 ng/ml, 7000 ng/ml, 7250 ng/ml, 7500 ng/ml, 7750 ng/ml, 8000 ng/ml, 8250 ng/ml, 8500 ng/ml, 8750 ng/ml, 9000 ng/ml, 9250 ng/ml, 9500 ng/ml, 9750 ng/ml, or about 10,000 ng/ml.

In one embodiment, the mitogenic growth factor, salt thereof, ester thereof, di- or tri-peptide thereof, or combination thereof has a concentration of about 0.1 ng/ml to about 1 ng/ml, about 0.2 ng/ml to about 1 ng/ml, about 0.3 ng/ml to about 1 ng/ml, about 0.4 ng/ml to about 1 ng/ml, about 0.5 ng/ml to about 1 ng/ml, about 0.6 ng/ml to about 1 ng/ml, about 1 ng/ml to about 5 ng/ml, about 1 ng/ml to about 4 ng/ml, about 1 ng/ml to about 3 ng/ml, about 1 ng/ml to about 2 ng/ml, about 1 ng/ml to about 10 ng/ml, about 1 ng/ml to about 20 ng/ml, about 1 ng/ml to about 50 ng/ml, about 1 ng/ml to about 100 ng/ml, about 1 ng/ml to about 200 ng/ml, about 1 ng/ml to about 500 ng/ml, about 1 ng/ml to about 1000 ng/ml; about 10 ng/ml to about 20 ng/ml, about 10 ng/ml to about 30 ng/ml, about 10 ng/ml to about 40 ng/ml, about 10 ng/ml to about 50 ng/ml, about 10 ng/ml to about 60 ng/ml, about 10 ng/ml to about 70 ng/ml, about 10 ng/ml to about 80 ng/ml, about 10 ng/ml to about 90 ng/ml, about 10 ng/ml to about 100 ng/ml; about 100 ng/ml to about 200 ng/ml, about 100 ng/ml to about 300 ng/ml, about 100 ng/ml to about 400 ng/ml, about 100 ng/ml to about 500 ng/ml, about 100 ng/ml to about 600 ng/ml, about 100 ng/ml to about 700 ng/ml, about 100 ng/ml to about 800 ng/ml, about 100 ng/ml to about 900 ng/ml, about 100 ng/ml to about 1000 ng/ml; about 1000 ng/ml to about 2000 ng/ml, about 1000 ng/ml to about 3000 ng/ml, about 1000 ng/ml to about 4000 ng/ml, about 1000 ng/ml to about 5000 ng/ml, about 1000 ng/ml to about 6000 ng/ml, about 1000 ng/ml to about 7000 ng/ml, about 1000 ng/ml to about 8000 ng/ml, about 1000 ng/ml to about 10000 ng/ml, about 2000 ng/ml to about 10000 ng/ml; about 5000 ng/ml to about 10000 ng/ml, about 300 ng/ml to about 1000 ng/ml, about 30 ng/ml to about 100 ng/ml, about 40 ng/ml to about 200 ng/ml, about 40 ng/ml to about 150 ng/ml, about 10 ng/ml to about 200 ng/ml, about 10 ng/ml to about 250 ng/ml, or about 50 ng/ml to about 500 ng/ml.

In one embodiment, the albumin, salt thereof, ester thereof, di- or tri-peptide thereof, or combination thereof has a concentration of about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 6%, 7%, 8%, 9%, or about 10%.

In one embodiment, the albumin, salt thereof, ester thereof, di- or tri-peptide thereof, or combination thereof has a concentration of about 0.1% to about 5%, about 0.15% to about 5%, about 0.2% to about 5%, about 0.25% to about 5%, about 0.3% to about 5%, about 0.35% to about 5%, about 0.4% to about 5%, about 0.45% to about 5%, about 0.5% to about 5%, about 0.55% to about 5%, about 0.6% to about 5%, about 0.65% to about 5%, about 0.7% to about 5%, about 0.75% to about 5%, about 0.8% to about 5%, about 0.85% to about 5%, about 0.9% to about 5%, about 0.95% to about 5%, about 1% to about 5%, about 1.05% to about 5%, about 1.1% to about 5%, about 1.15% to about 5%, about 1.2% to about 5%, about 1.25% to about 5%, about 1.3% to about 5%, about 1.35% to about 5%, about 1.4% to about 5%, about 1.45% to about 5%, about 1.5% to about 5%, about 1.55% to about 5%, about 1.6% to about 5%, about 1.65% to about 5%, about 1.7% to about 5%, about 1.75% to about 5%, about 1.8% to about 5%, about 1.85% to about 5%, about 1.9% to about 5%, about 1.95% to about 5%, about 2% to about 5%, about 0.1% to about 1%, about 0.05% to about 2.0%, about 0.1% to about 2%, about 0.15% to about 2%, about 0.2% to about 2%, about 0.25% to about 2%, about 0.3% to about 2%, about 0.35% to about 2%, about 0.4% to about 2%, about 0.45% to about 2%, about 0.5% to about 2%, about 0.55% to about 2%, about 0.6% to about 2%, about 0.65% to about 2%, about 0.7% to about 2%, about 0.75% to about 2%, about 0.8% to about 2%, about 0.85% to about 2%, about 0.9% to about 2%, about 0.95% to about 2%, about 1% to about 2%, about 1.05% to about 2%, about 1.1% to about 2%, about 1.15% to about 2%, about 1.2% to about 2%, about 1.25% to about 2%, about 1.3% to about 2%, about 1.35% to about 2%, about 1.4% to about 2%, about 1.45% to about 2%, about 1.5% to about 2%, about 1.55% to about 2%, about 1.6% to about 2%, about 1.65% to about 2%, about 1.7% to about 2%, about 1.75% to about 2%, or about 1.8% to about 2%. In some embodiments, the amount of albumin and amount of small molecule inhibitor (e.g., the GSK3 inhibitor) in the pluripotent stem cell composition is balanced for maintenance of pluripotency upon expansion. In such embodiments of the combination of the small molecule inhibitor and albumin, the small molecule inhibitor can be included throughout the culture period.

In one embodiment, the pluripotent stem cell composition or supplement composition comprises a combination of components, such as one or more small molecule inhibitors, mitogenic growth factors, albumins, salts thereof, esters thereof, di- or tri-peptides thereof, or combinations thereof wherein each component (or combinations thereof) are combined or separately added to a cell culture, culture vessel, or other container. For example, an aliquot of a stock solution of each small molecule inhibitor, mitogenic growth factor, or albumin may be combined to comprise the pluripotent stem cell composition, or supplement composition, or each may be added individually to a cell culture or other container. In one aspect, the pluripotent stem cell composition or supplement composition may comprise one or more small molecule inhibitors and one or mitogenic growth factors, salts thereof, esters thereof, or combinations thereof in addition to other cell culture compatible components. In another aspect, the pluripotent stem cell composition or supplement composition may comprise one or more small molecule inhibitors, one or mitogenic growth factors, and one or more albumins, salts thereof, esters thereof, di- or tri-peptides thereof, or combinations thereof in addition to other cell culture compatible components.

The exemplary medium and supplement components shown in Table 1 can be formulated into solutions, concentrates, powders, agglomerated powders, tablets, capsules, or other delivery means. In one aspect, the feed or supplement formulation is a solid powder or agglomerated powder. In some embodiments, the exemplary formulations can be formulated as a liquid or dry powder that is reconstituted, added directly to the culture, or dissolved in a cell culture compatible vehicle (e.g., water, buffer, media, feed solution, etc.) prior to use. In one aspect, the exemplary formulations are liquid. In another aspect, the exemplary formulations are liquid concentrate that is diluted prior to or during use. Another aspect is a cell culture or cell culture system that has been supplemented with the pluripotent stem cell composition or supplement composition components described herein either individually or collectively. Another aspect is a cell culture system that utilizes the pluripotent stem cell composition or supplement composition described herein. Another aspect is a method of increasing PSC growth, enhancing PSC proliferation, maintaining PSC pluripotency, maintaining spheroid morphology, maintaining PSC morphology, increasing PSC passage count, or increasing PSC culture scale, or a combination thereof by adding the pluripotent stem cell composition or supplement composition described herein to a cell culture or cell culture system. Another embodiment described herein is a cell culture that has increased PSC growth, enhanced PSC proliferation, maintained PSC pluripotency, maintained spheroid morphology, maintained PSC morphology, increased PSC passage count, or increased PSC culture scale or a combination thereof that has been administered the pluripotent stem cell composition or supplement composition described herein or the individual components thereof.

Nutritive media, media supplements and media subgroups produced as described herein are any media, media supplement or media subgroup (serum-free or serum-containing) which may be used to support the growth of a cell, which may be an animal cell (particularly a mammalian cell, most preferably a human cell), any of which may be a somatic cell, a germ cell, a normal cell, a diseased cell, a transformed cell, a mutant cell, a stem cell, a precursor cell or an embryonic cell. The cell may be a self-replicating cell such as a stem cell. The stem cell may be a pluripotent stem cell (PSC) such as an induced pluripotent cell (iPSC), embryonic stem cell (ES cells) derived from embryos, embryonic stem cells made by somatic cell nuclear transfer (ntES cells), and embryonic stem cells from unfertilized eggs (parthenogenesis embryonic stem cells, or pES cells). Such nutritive media may include, but are not limited to, cell culture media, preferably a PSC culture medium or animal cell culture medium. The PSC media and/or media supplements may include, but are not limited to, biological fluids (particularly animal sera, including without limitation bovine serum, particularly fetal bovine, newborn calf or normal calf serum, horse serum, porcine serum, rat serum, murine serum, rabbit serum, monkey serum, ape serum or human serum, any of which may be fetal serum) and extracts thereof (more preferably serum albumin including without limitation bovine serum albumin or human serum albumin). The PSC media and/or media supplements may include recombinantly expressed albumin, extracts orpeptide fragments thereof, such as recombinant human albumin. Medium supplements may also include defined replacements such as StemPro™ LipoMAX™ supplement, OptiMAb™ supplement, Knock-Out™ Serum Replacement (Gibco™, Thermo Fisher Scientific). Such supplements may also comprise defined components, including but not limited to, hormones, cytokines, neurotransmitters, lipids, attachment factors, proteins, inter alia.

In one embodiment, the feed or supplement comprises agglomerated powders of media, media supplements, media subgroups, or buffers. In one aspect described herein, the agglomerated media supplements are produced using fluid bed technology to agglomerate the solutions of media, media supplements, media subgroups, or buffers. Fluid bed technology is a process of producing agglomerated powders having altered characteristics (particularly, for example, solubility) from the starting materials. In general, applications of the technology, powders are suspended in an upwardly moving column of air while at the same time a controlled and defined amount of liquid is injected into the powder stream to produce a moistened state of the powder; mild heat is then used to dry the material, producing an agglomerated powder. In some aspects, the agglomerated media supplements or subgroups are produced using the proprietary Advanced Granulation Technology™ (AGT™ dry media format) (Gibco™). See Jayme et al., “A Novel Application of Granulation Technology to Improve Physical Properties and Biological Performance of Powdered Serum-Free Culture Media,” In: Shirahata et al. (Eds) Animal Cell Technology: Basic & Applied Aspects. Vol. 12 (2002), Springer, Dordrecht.

The formulations and methods described herein can be used to prepare nutritive media supplements or media supplement subgroups for increasing PSC growth, enhancing PSC proliferation, maintaining PSC pluripotency, maintaining spheroid morphology, maintaining PSC morphology, increasing PSC passage count, or increasing PSC culture scale, or a combination thereof.

Any nutritive medium, medium supplement, or medium supplement subgroup may be prepared by the methods described herein. Particularly nutritive media supplements or supplement subgroups that may be prepared as described herein include cell culture media, feeds, or supplements, and media supplement subgroups that support the growth of human and other animal cells, in particular stem cells such as, but not limited to, PSCs such as iPSCs, ES cells, ntES cells, and pES cells.

Examples of animal cell culture media that may be utilized as described herein include, but are not limited to, DMEM, RPMI-1640, MCDB 131, MCDB 153, MDEM, IMDM, MEM, M199, McCoy's 5A, Williams' Media E, Leibovitz's L-15 Medium, F10 Nutrient Mixture, F12 Nutrient Mixture, MEF (mouse embryonic fibroblast)-conditioned media, StemFlex, Nutristem hESC XF culture medium, Essential 8™ media, mTeSR media, mTeSR-2 media, and cell-specific serum-free media (SFM) such as those designed to support the culture of stem cells, PSCs, keratinocytes, endothelial cells, hepatocytes, melanocytes, various CHO cells, 293 cells, PerC6, hybridomas, hematopoetic cells, embryonic cells, neural cells etc. Specific chemically defined media products include CD CHO Medium (Gibco™), CD OptiCHO Medium (Gibco™) Dynamis Medium (Gibco™), ExpiCHO Stable Production Medium (Gibco™) BalanCD® CHO Growth A (Irvine Scientific), PowerCHO™ Advance (Lonza), EX-CELL® Advanced™ CHO Medium (Millipore Sigma-Aldrich), HyClone™ ActiPro™ (GE Healthcare Life Sciences). Specific feed supplements include CHO CD EfficeientFeed™ A (or B) AGT™ Nutritional Supplement (Gibco™), CD EfficientFeed™ C AGT™ Nutrient Supplement (Gibco®), EfficientFeed™ A+AGT™ Supplement (Gibco™), EfficientFeed™ B+AGT™ Supplement (Gibco™), Resurge™ CD1 Supplement (Gibco™), HyClone™ Cell Boost Supplements (various versions) (GE Healthcare Life Sciences), EX-CELL® Advanced™ CHO Feed 1 (Millipore Sigma-Aldrich), et al. Other media, media supplements, and media subgroups suitable for preparation are available commercially. Formulations for these media, media supplements and media subgroups, as well as many other commonly used animal cell culture media, media supplements and media subgroups are well-known in the art and are described in the literature and available from commercial suppliers, e.g., Thermo Fisher Scientific, Life Technologies, Gibco, Invitrogen, et al.

Any of the above media, media supplements, media supplement subgroups, or buffers that can be used as described herein may also include one or more additional components, such as indicating or selection agents (e.g., dyes, antibiotics, amino acids, enzymes, substrates and the like), filters (e.g., charcoal), salts, polysaccharides, ions, detergents, stabilizers, and the like.

In another embodiment described herein, the pluripotent stem cell composition or supplement composition may comprise one or more buffer salts at concentrations sufficient to provide optimal buffering capacity for the culture medium. In one aspect, the one or more buffer comprises acetic acid, acetylsalicylic acid, adipic acid, alginic acid, ascorbic acid, aspartic acid, benzoic acid, benzenesulfonic acid, bisulfic acid, boric acid, butanoic acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, gly-glycine, gluco heptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthilic acid, nicotinic acid, nitrous acid, oxalic acid, pelargonic, phosphoric acid, propionic acid, pyruvic acid, saccharin, salicylic acid, sorbic acid, succinic acid, sulfuric acid, tartaric acid, thiocyanic acid, thioglycolic acid, thiosulfuric acid, tosylic acid, undecylenic acid, MES, bis-tris methane, ADA, ACES, bis-tris propane, PIPES, MOPSO, cholamine chloride, MOPS, BES, TES, HEPES, DIPSO, MOBS, acetamido glycine, TAPSO, TEA, POPSO, HEPPSO, EPS, HEPPS, Tricine, Tris(hydroxymethyl)aminomethane (tromethamine), glycinamide, glycylglycine, HEPBS, Bicine, TAPS, AMPB, CHES, AMP, AMPSO, CAPSO, CAPS, CABS, combinations thereof, or salts thereof. In one aspect, the buffer comprises one or more of phosphate, sulfate, carbonate, formate, acetate, propionate, butanoate, lactate, glycine, maleate, pyruvate, citrate, aconitate, isocitrate, α-ketoglutarate, succinate, fumarate, malate, oxaloacetate, aspartate, glutamate, tris(hydroxymethyl)aminomethane (tromethamine), combinations thereof, or salts thereof.

In one aspect described herein, a buffer salt, such as sodium bicarbonate, may be added to the cell culture medium, feed, or supplement prior to, during, or following agglomeration of the medium. In one example of this aspect described herein, the buffer salt may be added to the culture medium prior to, during or following agglomeration with an appropriate solvent (such as water, serum or a pH-adjusting agent such as an acid (e.g., HCl at a concentration of 1 M to 5 M, 0.1 M to 5 M, or preferably at 1 M) or a base (e.g., NaOH at a concentration of 1 M to 5 M, 0.1 M to 5 M, or preferably at 1 M) such that, upon reconstitution of the agglomerated medium the culture medium is at the optimal or substantially optimal pH for cultivation of a variety of cell types. For example, animal cell culture media prepared by the present methods will, upon reconstitution, preferably have a pH of about 6-8 or about 7-8, more preferably about 7-7.5, or about 7.2-7.4.

In another example, one or more buffer salts may be added directly to a nutritive medium. In a related aspect, a pH-adjusting agent such as an acid (e.g., HCl) or a base (e.g., NaOH) may be added to a nutritive medium, which may contain one or more buffer salts, by agglomeration of the pH-adjusting agent into nutritive medium in a fluid bed apparatus, by spray-drying the pH-adjusting agent onto the powdered or agglomerated nutritive medium, or by a combination thereof; this approach obviates the subsequent addition of a pH-adjusting agent after reconstitution of the powdered medium. The nutritive culture medium described herein is useful in cultivation or growth of cells in vitro that, upon reconstitution with a solvent (e.g., water or serum), has a pH that is optimal for the support of cell cultivation or growth without a need for adjustment of the pH of the liquid medium. For example, a mammalian cell culture medium prepared according to these methods may have a pH of between about 7.1 to about 7.5, more preferably between about 7.1 to about 7.4, and most preferably about 7.2 to about 7.4 or about 7.2 to about 7.3.

In another embodiment, pH-opposing forms of certain media components (particularly phosphate or other buffer salts) may be used in the culture medium to provide a desired pH. pH-opposing forms of components are conjugate acid-base pairs in which the members of the pair can either raise the pH or lower it to achieve the desired pH of the solution. Sodium HEPES (pH raising) and HEPES-HCl (pH lowering) are examples of pH opposing components. For example, if a media having a pH of between 4.5 and 7.2 is to be prepared, the first step is to determine the correct balance of monobasic (to lower the pH) to dibasic (to raise the pH) phosphate in order to yield the desired pH. Typically, mono- and di-basic phosphate salts are used at concentrations of about 0.1 mM to about 10 mM, about 0.2 mM to about 9 mM, about 0.3 mM to about 8.5 mM, about 0.4 mM to about 8 mM, about 0.5 mM to about 7.5 mM, about 0.6 mM to about 7 mM, or preferably about 0.7 mM to about 7 mM. If other buffer systems are used in the formulations, the proper ratio or balance of the basic (typically sodium or monobasic) buffer salt and the corresponding acidic (or pH-opposing; typically HCl or dibasic) buffer salt is similarly determined to ensure that the formulation will be at the desired final pH. Because the actual phosphate molecular species that is present in a solution is the same at a given pH whether the basic (e.g., sodium or monobasic) or acidic (e.g., HCl or dibasic) form is added, this adjustment would not be expected to impact buffering capacity. Once an appropriate ratio of pH-opposing forms of an appropriate buffer is determined, these components may be added to the medium (for example, a dry powder medium) to provide a culture medium that is of the appropriate pH level prior to use.

Another embodiment is a pluripotent stem cell composition or supplement composition as described herein that directly support the cultivation of cells in vitro, without the need for the addition of any supplemental nutrient components to the medium prior to use. Media according to this aspect described herein thus will preferably comprise the nutritional components necessary for cultivation of a cell in vitro, such that no additional nutritional components need be included in the solvent or added to the medium prior to use. Such complete media may be automatically pH-adjusting media, and may comprise one or more components such as one or more culture medium supplements (including but not limited to serum, serum replacement supplement or recombinant albumin), one or more amino acids (including but not limited to 1-glutamine), insulin, transferrin, one or more hormones, one or more lipids, one or more growth factors, one or more mitogenic growth factors, one or more small molecule inhibitors, one or more cytokines, one or more neurotransmitters, one or more extracts of animal tissues, organs or glands, one or more enzymes, one or more proteins, one or more trace elements, one or more extracellular matrix components, one or more antibiotics, one or more viral inhibitors, and or one or more buffers. In some embodiments, the complete media can further be supplemented with feeds or supplements comprising at least one small molecule inhibitor, mitogenic growth factor, or combinations thereof as the cells grow and consume or deplete such components from the media. In certain embodiments, the complete media can further be supplemented with feeds or supplements comprising at least one small molecule inhibitor, mitogenic growth factor, and albumin, salts thereof, esters thereof, di or tri peptides thereof, or combinations thereof as the cells grow and consume or deplete such components from the media. In some embodiments, the pluripotent stem cell composition, supplement and/or complete media is serum-free. In other embodiments, the pluripotent stem cell composition, supplement and/or complete media is animal origin free.

Examples of additional components that may be added to the media, feeds or supplements described herein, or that may be prepared by the methods described herein, include, without limitation, animal sera, such as bovine sera, fetal bovine, newborn calf and calf sera, human sera, equine sera, porcine sera, monkey sera, ape sera, rat sera, murine sera, rabbit sera, ovine sera and the like, defined replacements such as StemPro™ LipoMAX™ supplement, OptiMAb™ supplement, Knock-Out™ Serum Replacement (Gibco™, Thermo Fisher Scientific), hormones (including steroid hormones such as corticosteroids, estrogens, androgens (e.g., testosterone) and peptide hormones such as insulin, cytokines (including growth factors (e.g., EGF, αFGF, βFGF, HGF, IGF-1, IGF-2, NGF and the like), interleukins, colony-stimulating factors, interferons and the like), mitogenic growth factors (e.g., EGF, TGF-α, TGF-β, bFGF, BDNF, HGF, HRG, KGF, VEGF, PDGF, IGF, MSF, SGF, NGF, FGF, G-CSF, GM-CSF, Erythropoetin, TPO, BMP, GDF, Neurotrophins, SGF), small molecule inhibitors (e.g., GSK3 inhibitors, rho kinase inhibitors), neurotransmitters, lipids (including phospholipids, sphingolipids, fatty acids, Excyte™, cholesterol and the like), attachment factors (including extracellular matrix components such as fibronectin, vitronectin, laminins, collagens, proteoglycans, gly cosaminogly cans and the like), and extracts or hydrolysates of animal tissues, cells, organs or glands (such as bovine pituitary extract, bovine brain extract, chick embryo extract, bovine embryo extract, chicken meat extract, chicken tissue extract, achilles tendon and extracts thereof) and the like). Other media supplements that may be produced by the present methods or that may be included in the culture media described herein include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin, or dispase); lipoproteins; fetuin; ferritin; etc.), which may be natural or recombinant; vitamins; amino acids and variants thereof (including, but not limited to, 1-glutamine and 1-cystine), enzyme co-factors; polysaccharides; salts or ions (including trace elements such as salts or ions of molybdenum, vanadium, cobalt, manganese, selenium, and the like); and other supplements and compositions that are useful in cultivating cells in vitro that will be familiar to one of ordinary skill. In some embodiments, media and/or media supplements produced by the methods described herein include animal or mammalian (e.g., human, fish, bovine, porcine, equine, monkey, ape, rat, murine, rabbit, ovine, insect, etc.) derived supplements, ingredients, or products. These sera and other media supplements are available commercially. Alternatively, sera and other media supplements described herein may be isolated from their natural sources or produced recombinantly by art-known methods that will be routine to one of ordinary skill. See Freshney, R. I., Culture of Animal Cells, New York: Alan R. Liss, Inc., pp. 74-78 (1983); see also Harlow, E., and Lane, D., Antibodies: A Laboratory Manual, Cold Spring Harbor Laboratory, N.Y. (1988). In other embodiments, media and/or media supplements produced by the methods described herein do not include animal derived components and are animal-origin free media and/or supplements.

Examples of buffers that may be used in conjunction with the pluripotent stem cell compositions or supplement compositions described herein include, but are not limited to, buffered saline solutions, phosphate-buffered saline (PBS) formulations, Tris-buffered saline (TBS) formulations, HEPES-buffered saline (HBS) formulations, Hanks' Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, and Kao and Michayluk's Basal Salt Solutions, and the like. Formulations for these buffers, which are commercially available, as well as for many other commonly used buffers, are well-known in the art and may be found for example in the Thermo Fisher Scientific Catalogue, in the DIFCO™ & BBL™ Manual, 2nd ed. (Becton, Dickinson and Company, 2009), and in the Millipore Sigma Cell Culture Catalogue.

One method for determining the effective concentration of a compound (e.g., a vitamin) in a test culture medium is as follows. Using a vitamin for the purposes of illustration, a known concentration of the vitamin is serially diluted into a culture medium lacking the vitamin. A second set of serial dilutions are set-up where the test culture medium is serially diluted into a culture medium also lacking the vitamin. Cells that require the vitamin for growth are then added to both sets of serially diluted samples and cultured under appropriate conditions. After a period of time, cell replication is measured (e.g., by cell counting or by measuring optical density). The measurements of the known concentrations are graphed to form a standard curve, to which the measurements from the test culture medium dilutions are compared to determine the effective concentration of the vitamin in the test culture medium. Any number of similar assays may be used to determine the amounts of metabolites in a sample.

Another embodiment described herein is a method for sterilizing the nutritive media, media supplements, media supplement subgroups or buffers described herein, as well as for sterilizing powdered nutritive media, media supplements, media subgroups and buffers prepared by standard methods such as ball-milling or lyophilization. Also described are methods for sterilizing or substantially sterilizing the samples including nutritive media, media supplements, media subgroups, and buffers described herein. Such additional methods may include filtration, heat sterilization, irradiation, or other chemical or physical methods. Nutritive media, media supplements, media subgroups, or buffers (prepared as described herein may be irradiated under conditions favoring sterilization. Since nutritive media, media supplements, media subgroups, and buffers are usually prepared in large volume solutions and frequently contain heat labile components, they are not amenable to sterilization by irradiation or by heating. Thus, nutritive media, media supplements, media subgroups, and buffers are commonly sterilized by contaminant-removal methods such as filtration, which significantly increases the expense and time required to manufacture such media, media supplements, media subgroups, and buffers.

Nutritive media, media supplements, media supplement subgroups, or buffers prepared according to the methods described herein can be sterilized by common methods in the art including filtration, irradiation, or autoclaving. For example, nutritive media, media supplements, media subgroups, or buffers may be irradiated under conditions favoring sterilization. Preferably, this irradiation is accomplished in bulk (i.e., following packaging of the sample, nutritive media, media supplement, media subgroup, or buffer), and most preferably this irradiation is accomplished by exposure of the bulk packaged sample, media, media supplement, media subgroup, or buffer described herein to a source of gamma rays under conditions such that bacteria, fungi, spores or viruses that may be resident in the nutritive media, media supplements, media subgroups, or buffers are inactivated (i.e., prevented from replicating). Alternatively, irradiation may be accomplished by exposure of the sample, nutritive media, media supplement, media subgroup, or buffer, prior to packaging, to a source of gamma rays or a source of ultraviolet light. The sample, media, media supplements, media subgroups and buffers described herein may alternatively be sterilized by heat treatment (if the subgroups, or components of the sample, nutritive media, media supplement, media subgroup, or buffer are heat stable), for example by flash pasteurization or autoclaving. As will be understood by one of ordinary skill in the art, the dose of irradiation or heat, and the time of exposure, required for sterilization will depend upon the bulk of the materials to be sterilized, and can easily be determined by those of ordinary skilled in the art without undue experimentation.

The nutritive media, media feeds or supplements, media supplement subgroups, or buffers may be used to culture or manipulate cells according to standard cell culture techniques that are known to one of ordinary skill in the art. In such techniques, the cells to be cultured are contacted with the nutritive media, media supplement, media subgroup, or buffer described herein under conditions favoring the cultivation or manipulation of the cells (such as controlled temperature, humidity, cell agitation, lighting, and atmospheric conditions). Cells that are particularly amenable to cultivation by such methods include, but are not limited to, animal cells. Such animal cells are available commercially from known culture depositories, e.g., the American Type Culture Collection (ATCC, Manassas, Va.) and others that will be familiar to one of ordinary skill in the art. In some embodiments, the animal cells for cultivation by these methods include, but are not limited to, mammalian cells (such as CHO cells, COS cells, VERO cells, BHK cells, AE-1 cells, SP2/0 cells, L5.1 cells, hybridoma cells and human cells, such as 293 cells, PER-C6 cells and HeLa cells), any of which may be a somatic cell, a germ cell, a normal cell, a diseased cell, a transformed cell, a mutant cell, a stem cell, a PSC, a precursor cell or an embryonic cell, embryonic stem cells (ES cells), an IPSC, an ntES cell, a pES cell, cells used for virus or vector production (i.e., 293, PerC 6), cells derived from primary human sites used for cell or gene therapy, i.e., lymphocytes, hematopoietic cells, other white blood cells (WBC), macrophage, neutrophils, dendritic cells, and any of which may be an anchorage-dependent or anchorage-independent (i.e., “suspension”) cell. Another aspect is the manipulation or cultivation of cells and/or tissues for tissue or organ transplantation or engineering, i.e., hepatocyte, pancreatic islets, osteoblasts, osteoclasts/chondrocytes, dermal or muscle or other connective tissue, epithelial cells, tissues like keratinocytes, cells of neural origin, cornea, skin, organs, and cells used as vaccines, i.e., blood cells, hematopoietic cells other stem cells or progenitor cells, and inactivated or modified tumor cells of various histotypes.

Another embodiment is a method of manipulating or culturing one or more cells comprising contacting said cells with the feeds or supplements described herein, and incubating said cell or cells under conditions favoring the cultivation or manipulation of the cell or cells. Any cell may be cultured or manipulated according to the present methods, animal cells and other cells or cell lines described herein. Cells cultured or manipulated according to this aspect described herein may be normal cells, diseased cells, transformed cells, mutant cells, somatic cells, germ cells, stem cells, precursor cells, stem cells, PSCs or embryonic stem cells, iPSCs, any of which may be established cell lines or obtained from natural sources.

In one aspect, the PSC medium or supplement includes one or more amino acids. In another aspect, a salt of an amino acid is used. In another aspect, the salt is a sodium salt. In another aspect, monobasic and dibasic phosphate salts are used. A preferred cation is sodium. In another aspect, the monobasic and dibasic salts are provided such that a resultant pH, for example, about pH 7 is obtained. Depending on the formulation, while the ratio of monobasic to dibasic salts may be dictated by desired pH, different total salt concentrations should be tried to optimize solubility, especially when concentrated or highly concentrated supplements are to be used. The pH can also be confirmed when assessing the salt concentration. When an amino acid is not provided as a salt, the pH effect of the acid may be countered by a tribasic phosphate, preferably a sodium tribasic phosphate. While sodium is may be used as a cation, other metals, such as potassium, calcium, magnesium may be used. If a specific counter ion is desired, it may be available as a phosphate salt. In another aspect, the supplement can be prepared and used as a highly concentrated mixture, for example, with one or more components at a concentration about 2× or more, 3×, 5×, 8×, 10×, 12×, 15×, 20×, 25×, 50×, 75×, 85×, 95×, or even about 100× or more times the concentration of that component in the medium being supplemented. The concentration of each desired ingredient of the supplement can be independently selected.

A supplement may have no ingredients in common with the medium being supplemented or may have one or more ingredients in common. The supplement may differ from the medium being supplemented in at least one manner, such as a different concentration of one or more ingredients, for example a different ratio of two ingredients, a different ingredient mix, additional ingredients, or omitted ingredients in the supplement. For example, a supplement may omit salts to the extent feasible and may contain, for example, significantly enhanced concentrations of growth factors or amino acids. A preferred supplement formulation contains at least 2, more preferably 3, but perhaps at least 4, 5, 6, 7, 8, 9, 10, 11, 12, 13, 14, 15, or more amino acids including salts, or dimers thereof.

In some embodiments, PSC media or supplements as described herein are utilized to supplement a medium that has or is being used to culture cells, e.g., as the cells are cultured, some ingredients are removed from the medium by the cells. In some embodiments described herein, the feed supplement is used, inter alia, to replace some or all of these ingredients. In some embodiments, the PSC medium or supplement contains the majority of the ingredients that were in the original medium to be supplemented, but the PSC medium or supplement is lacking at least one ingredient. In some embodiments, the PSC medium or supplement is lacking 1, 2, 3, 4, 5, 6, 7, 8, 9, 10, 11, 12, 13, 14, 15, or more ingredients as compared to the concentration in the original culture medium being supplemented. In some embodiments, the PSC medium or supplement is added in a concentrated form, e.g., at 2×, 3×, 4×, 5×, 6×, 7×, 8×, 9×, 10×, 15×, 20×, 30×, 40×, 50×, 100×, 200×, 300×, 400×, 500× or 1000×. Concentrated form is meant that at least one of the ingredients in the PSC medium or supplement is at a concentration higher than what is the desired concentration in the culture medium. In some embodiments, the PSC medium is added to the cells in culture as a 1× formulation. In some embodiments, ingredients for a PSC medium or supplement may be divided into multiple PSC media or supplements, e.g., based upon compatible subgroups.

Osmolality (a measure of osmotic pressure) of cell culture medium is important as it helps regulate the flow of substances in and out of the cell. It is typically controlled by the addition or subtraction of salt in a culture medium. Rapid increases in osmolality (e.g., addition of concentrated supplement with elevated osmolality relative to the base growth medium) may result in stressed, damaged, or dead cells. Maintaining an optimal osmolality range during cell culture/growth is desirable for cell function and/or bioproduction success.

Base growth medium osmolality generally ranges from 250 mOsmo/kg to 350 mOsmo/kg. In some embodiments, addition of a concentrated PSC medium or supplement described herein increases osmolality by about 25 mOsmo/kg or by between from about 0 to about 100, about 0.01 to about 100, about 0.1 to about 100, about 1 to about 100, about 10 to about 100, about 50 to about 100, about 75 to about 100, about 1 to about 10, about 1 to about 50, about 1 to about 75, about 10 to about 50, about 15 to about 35, about 25 to about 50, or about 20 to about 30 mOsmo/kg. In some embodiments, the osmolality of a concentrated supplement medium described herein (e.g., a 5× concentrated supplement medium) has an osmolality between from about 0 to about 1500; 1 to about 1000; 1 to about 750; 1 to about 500; 1 to about 400; 1 to about 300; 1 to about 200; 1 to about 100; 1 to about 50; 50 to about 1000; 100 to about 1000; 300 to about 1000; 500 to about 1000; 750 to about 1000; 100 to about 200; 200 to about 300; 300 to about 400; 400 to about 500; 450 to about 500; 500 to about 600; 550 to about 650; 600 to about 700; 750 to about 850; 700 to about 800; 800 to about 900; 900 to about 1000; 1000 to about 1250; or about 1250 to about 1500 mOsmo/kg. In some embodiments, the osmolality of a concentrated PSC medium or supplement described herein is between from about 3.0× to about 3.5×, about 3.5× to about 4.5×, about 4.5× to about 5.5×, about 5.5× to about 6.5×, about 6.5× to about 7.5×, about 7.5× to about 8.5×, about 8.5× to about 9.5×, about 9.5× to about 10.5×, about 10.5× to about 11.5×, about 11.5× to about 12.5×, about 12.5× to about 13.5×, about 13.5× to about 14.5×, about 14.5× to 18.5 to about 19.5×, about 19.5× to about 20.5×, about 3× to about 10×, about 5× to about 10×, about 10× to about 15×, about 15× to about 20×, about 20× to about 25×, or about 25× to about 100× as compared to the osmolality of the medium being supplemented or fed.

In one embodiment provided herein is a pluripotent stem cell (PSC) composition comprises: a cell culture basal medium; a small molecule GSK3 inhibitor salts thereof, or esters thereof; a ROCK inhibitor, salts thereof, or esters thereof; and one or more mitogenic growth factors; wherein the composition may provide one or more of enhancing PSC growth, enhancing PSC proliferation, maintaining PSC pluripotency, maintaining spheroid morphology, maintaining PSC morphology, increasing PSC passage count, or increasing PSC culture scale as compared to customary PSC media. In another embodiment, the PSC composition further comprises one or more albumins or peptides thereof.

In one aspect, the GSK3 inhibitor of the PSC composition may include one or more of CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), BIO ((2′Z,3′E)-6-bromoindirubin-3′-oxime), AR-A 014418 (N-[(4-methoxyphenyl)methyl]-N′-(5-nitro-2-thiazolyl)urea)0, Kenpaullone (9-bromo-7,12-dihydro-indolo[3,2-d][1]benzazepin-6(5H)-one), SB 216763 (dichlorophenyl)-4-(1-methyl-1H-indol-3-yl)-1H-pyrrole-2,5-dione), or SB 415286 (3-[(3-chloro-4-hydroxyphenyl)amino]-4-(2-nitrophenyl)-1H-pyrrole-2,5-dione). In another aspect, the GSK3 inhibitor may be CHIR99021. As shown in Example 2, addition of CHIR99021 to the basal medium results in a significant improvement in fold-expansion of pluripotent stem cells. In some aspects, the GSK3 inhibitor may be added to the basal medium for the first day of culture only (for example, at the time of passage) or may be added to the basal medium continuously throughout the culture period. In some embodiments, culturing the PSCs continuously in the presence of the GSK3 inhibitor improves expansion fold change in suspension culture and maintenance of pluripotency As shown in Example 2, the continuous presence of the GSK3 inhibitor (e.g., CHIR99021) improves the fold-change of cells in suspension culture, but with some media formulations containing combinations of small molecule inhibitors, the spheroidal morphology is lost indicating pluripotency is lost. In such formulations, addition of CHIR99021 on Day 1 only followed by complete medium without CHIR99021 on Days 2-4 feeding supports pluripotent stem cell expansion. and maintenance of normal spheroidal morphology.

In one aspect, the rho kinase inhibitor of the PSC composition may inhibit ROCK1 activity and/or ROCK2 activity. The rho kinase inhibitor may be Y-27632 ((R)-(+)-trans-4-(1-aminoethyl)-N-(4-pyridyl)cyclohexanecarboxamide), Chroman 1, Emricasan, Polyamines, Trans-ISRIB, Pinacidil, or thiazovavin. In another aspect, the rho kinase inhibitor may be Y-27632 ((R)-(+)-trans-4-(1-aminoethyl)-N-(4-pyridyl)cyclohexanecarboxamide). Implementation of Y-27632 in conjunction with use of CHIR99021 at the time of seeding or passaging PSCs is shown to result in optimal fold change in Example 2.

In another aspect, the one or more mitogenic growth factors of the PSC composition includes one or more of EGF, TGF-α, TGF-β, bFGF, BDNF, HGF, HRG, and KGF. In another aspect, the PSC composition includes at least two mitogenic growth factors selected from EGF, TGF-α, TGF-β, bFGF, BDNF, HGF, HRG, and KGF. In another aspect, the PSC composition includes at least two mitogenic growth factors which comprise: EGF and TGF-α, EGF and TGF-β, EGF and bFGF, EGF and BDNF, EGF and HGF, EGF and HRG, EGF and KGF, TGF-α and TGF-β, TGF-α and bFGF, TGF-α and BDNF, TGF-α and HGF, TGF-α and HRG, TGF-α and KGF, TGF-β and bFGF, TGF-β and BDNF, TGF-β and HGF, TGF-β and HRG, TGF-β and KGF, bFGF and BDNF, bFGF and HGF, bFGF and HRG, bFGF and KGF, BDNF and HGF, BDNF and HRG, BDNF and KGF, HGF and HRG, HGF and KGF, or HRG and KGF. In another aspect, the composition may further comprise insulin and/or transferrin. In another aspect, the composition may further comprise one or more extracellular matrix components or proteins such as those selected from fibronectin, laminin, nidogen, collagen, vitronectin, or heparan sulfate proteoglycans, or fragments thereof. In some embodiments, the extracellular matrix proteins or fragments thereof are recombinant proteins or fragments, or synthetic peptides In another embodiment, the cell culture basal medium provided herein for PSC suspension culture may include amino acids, carbohydrates, vitamins, minerals, fatty acids, trace elements, antioxidants, salts, nucleosides, buffering agents, peptides, or a combination thereof.

In another aspect, the PSC culture medium may include albumin such as albumin derived from human (HSA), bovine (BSA), fetal bovine (FBS), rat, mouse, horse, monkey, or pig sera, or recombinant albumin or fragments thereof. In another aspect, the albumin may be HSA, BSA, FBS, peptides thereof, or combinations thereof. As shown in Example 3, inclusion of albumin at high concentrations together with CHIR99021 resulted in significant PSC expansion and maintenance of the pluripotency of the expanded PSCs.

As shown in Example 3, the medium and workflow as described herein supports expansion and pluripotency of iPSCs and ESCs. In another aspect, the PSC may be an iPSC or ESC. The PSC may be, for example, Gibco Episomal iPSCs, WTC-11, WA09, or WA01 cells.

The certain embodiments, PSC composition and supplement for suspension culture may contain additional components as described herein including, but not limited to pH indicators, antibiotics, carbohydrates, vitamins, minerals, fatty acids, trace elements, antioxidants, nucleosides, buffering agents, peptides, surfactants, non-essential amino acids, lipids, inorganic salts, organic salts, antimycotics, purine derivatives, solvents, buffers, sugars, hormones, additional growth factors, cytokines, antiviral agents, hormones, neurotransmitters, and attachment factors.

In another aspect, the composition may include amino acids that may comprise one or more of glycine, alanine, arginine, asparagine, aspartic acid, cysteine, cystine, glutamic acid, glutamine, histidine, isoleucine, leucine, lysine, methionine, phenylalanine, proline, serine, threonine, tryptophan, tyrosine, valine, salts thereof, esters thereof, or di- or tri-peptides thereof.

In another aspect, the composition may include vitamins that may comprise one or more of biotin (B7), choline, folic acid (B9), niacinamide (B3), pyridoxine (B6), riboflavin (B2), thiamine (B1), cobalamin (B12), inositol, retinol (A), pantothenic acid (B5), ascorbic acid (C), cholecalciferol (D), tocopherol (E), phylloquinone (K), lipoic acid, linoleic acid, para-aminobenzoic acid, salts thereof, or esters thereof.

In another aspect, the composition may include carbohydrates that may comprise one or more of glucose, sucrose, galactose, fructose, trehalose, pyruvate (e.g., sodium pyruvate).

In another aspect, the composition may include minerals that may comprise one or more of biotin, iron, manganese, copper, iodine, zinc, cobalt, fluoride, chromium, molybdenum, selenium, nickel, silicon, vanadium, salts thereof, or combinations thereof.

In another aspect, the composition may include fatty acids that may comprise one or more of fatty acids of the n-3, n-6 and n-9 families such as, but not limited to caprylic acid, capric acid, lauric acid, myristic acid, palmitic acid, stearic acid, arachidonic acid, linoleic acid, linolenic acid, oleic acid, palmitoleic acid, cholesterol synthetic, d/l-tocopherol acetate, behenic acid, lignoceric aid, cerotic acid, myristoleic acid, sapienic acid, elaidic acid, vaccenic acid, α-linolenic acid, erucic acid, eicosapentaenoic acid, or docosahexaenoic acid, or esters thereof, or derivatives thereof.

In another aspect, the composition may include inorganic or organic salts that may comprise one or more of an aluminum salt, a barium salt, a cadmium salt, a copper salt, a magnesium salt, a manganese salt, a nickel salt, a potassium salt, a calcium salt, a silver salt, a tin salt, a zirconium salt, a sodium salt, or combinations thereof. Salts include those made with organic or inorganic anions including, without limitation: AgNO₃, AlCl₃, Ba(C₂H₃O₂)₂, CaCl₂, CdSO₄, CdCl₂, CoCl₂, Cr₂(SO₄)₃, CuCl₂, CuSO₄, FeSO₄, FeCl₂, FeCl₃, Fe(NO₃)₃, GeO₂, Na₂SeO₃, H₂SeO₃, KBr, KCl, KI, MgCl₂, MgSO₄, MnCl₂, NaCl, NaF, Na₂SiO₃, NaVO₃, Na₃VO₄, (NH₄)₆Mo₇O₂₄, Na₂HPO₄, NaH₂PO₄, NaHCO₃, NiSO₄, NiCl₂, Ni(NO₃)₂, RbCl, SnCl₂, ZnCl₂, ZnSO₄, ZrOCl₂, EDTA tetrasodium, pyridoxine HCL, or combinations thereof. In one aspect, the composition comprises one or more salts selected from the group consisting of: calcium chloride, cupric sulfate, ferric nitrate, ferric sulfate, magnesium chloride, magnesium sulfate, potassium chloride, potassium iodide, sodium chloride, sodium phosphate dibasic, sodium phosphate monobasic, zinc sulfate, pyridoxine HCl, sodium, potassium, magnesium, calcium, ammonium, phosphate, carbonate, bicarbonate, sulfate, citrate, acetate, nitrate, ions of any of the foregoing, and any combination thereof.

In another aspect, the composition may include antioxidants that may comprise one or more of d/l-Lipoic Acid Thioctic, ascorbic acid 2 phosphate, dithiothreitol (DTT), vitamin A, vitamin E, vitamin K3, vitamin D2, calciferol, niacin, niacinamide, ascorbic acid, tocopherol, ascorbate, N-acetyl-1-cysteine (NAC), or glutathione reduced.

In another aspect, the composition may include nucleosides that may comprise one or more of adenosine, guanosine, thymidine, cytidine, uridine, xanthosine, or inosine.

In another aspect, the composition may include buffering agents that may comprise one or more of sodium bicarbonate, phosphate, sulfate, HEPES, PIPES, MOPS, MES, sodium phosphate dibasic, or sodium phosphate monobasic.

In another aspect, the composition may include surfactants that may comprise one or more of Pluronic F68, (polyoxyethylene-polyoxypropylene block copolymer) Synperonics (poly(ethylene glycol)-block-poly(propylene glycol)-block-poly(ethylene glycols)), Pluronic F127 (polyoxypropylenepolyoxyethylene block copolymer), Kolliphor® (polyethoxylated castor oil polysorbate 80 (Tween® 80), polyethylene glycol (PEG) 8,000, PEG 20,000, or Cremophor® EL (polyethoxylated castor oil), or combinations thereof.

Another embodiment described herein is a method for growing pluripotent stem cells (PSCs) in suspension, the method may comprise: providing pluripotent stem cells to be cultured in suspension; and culturing a PSC in suspension under conditions favorable for growth in any of the PSC compositions as described herein; wherein after at least 1 day after culturing the cells, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition may be exchanged. At least 25% of the composition may be exchanged daily. In another aspect, at least 50% of the medium and supplement composition may be exchanged daily. The composition may be exchanged every other day. As shown in Examples 3 and 4, the medium compositions and supplement compositions described herein support PSC increased fold change in expansion and maintenance of pluripotency when the composition is exchanged daily or exchanged every other day.

Another embodiment described herein is a method for maintaining pluripotency of pluripotent stem cells (PSCs), the method may comprise: contacting a PSC with a medium that may comprise: any of the PSC media compositions or supplement compositions as described herein; wherein after at least 1 day after contacting the cells, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition may be exchanged. At least 25% of the composition may be exchanged daily. In another aspect, at least 50% of the medium and supplement composition may be exchanged daily. The composition may be exchanged every other day.

Another embodiment described herein is a method for maintaining cellular morphology of pluripotent stem cells (PSCs), the method may comprise: contacting a PSC with a medium that may comprise: any of the PSC media compositions or supplement compositions as described herein; wherein after at least 1-day after contacting the cells, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition may be exchanged. At least 25% of the composition may be exchanged daily. In another aspect, at least 50% of the medium and supplement composition may be exchanged daily. The composition may be exchanged every other day.

Another embodiment described herein is a pluripotent stem cell that may be cultivated using the process of any of the processes as described herein.

Another embodiment as described herein is a means for growing pluripotent stem cells (PSCs), the process may comprise: providing pluripotent stem cells to be cultured in suspension; culturing a PSC in suspension under conditions favorable for growth in any of the PSC compositions as described herein; wherein after at least 1-day after culturing the cells, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition may be exchanged. At least 25% of the composition may be exchanged daily. In another aspect, at least 50% of the medium and supplement composition may be exchanged daily. The composition may be exchanged every other day.

Another embodiment described herein is a means for maintaining pluripotency of pluripotent stem cells (PSCs), the process may comprise: contacting a PSC with a medium that may comprise: any of the PSC media compositions or supplement compositions as described herein; wherein after at least 1-day after contacting the cells, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition may be exchanged. At least 25% of the composition may be exchanged daily. In another aspect, at least 50% of the medium and supplement composition may be exchanged daily. The composition may be exchanged every other day.

Another embodiment described herein is a means for maintaining cellular morphology of pluripotent stem cells (PSCs), the process may comprise: contacting a PSC with a medium that may comprise: any of the PSC media compositions or supplement compositions as described herein; wherein after at least 1 day after contacting the cells, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the medium and supplement composition may be exchanged. At least 25% of the composition may be exchanged daily. In another aspect, at least 50% of the medium and supplement composition may be exchanged daily. The composition may be exchanged every other day.

Another embodiment described herein is a use of a PSC composition provided herein for culturing pluripotent stem cells (PSCs) that may comprise: culturing a PSC under conditions favorable for growth in a composition that may comprise: any of the PSC compositions or stem cell culture supplement compositions as described herein. In one aspect, provided herein is a use of a PSC composition provided herein for culturing PSCs in suspension culture such that the PSC composition provides one or more of enhancing PSC growth, enhancing PSC proliferation, maintaining PSC pluripotency, maintaining spheroid morphology, maintaining PSC morphology, increasing PSC passage count, or increasing PSC culture scale as compared to customary PSC media.

Another embodiment described herein is a cell culture feed or supplement that may be prepared by the process of any of the processes as described herein.

Another embodiment described herein is a kit for culturing pluripotent stem cells (PSCs), the kit may comprise: one or more containers containing dry or liquid solutions of the media, media supplements, and cell-containing compositions described herein. Such a kit may comprise one or more containers such as vials, test tubes, bottles, packages, pouches, drums, and the like. Each of the containers may contain one or more of the cell culture reagents, nutritive media, media supplements, or cells described herein, or combinations thereof. Cell culture reagents, nutritive media, or media supplements may be hydrated or dehydrated. Such preparations may be sterile or substantially sterile. The kits may further comprise one or more additional containers containing a solvent to be used in reconstituting the dry powder pharmaceutical or clinical compositions, cell culture reagents, nutritive media, media supplements, media subgroups and/or buffers; such solvents may be aqueous or organic and include buffer solutions, saline solutions, nutritive medium solutions, or combinations thereof.

In some embodiments, a kit may comprise a container containing a PSC medium and/or supplement, instructions for use and means for accessing the medium or supplement such as a tear strip. The kits may also contain, in one or more additional containers, one or more cells such as PSCs or PSC lines.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1 PSC Medium Compositions and Culture

The following is an exemplary protocol for use of the PSC medium formulations provided herein for expanding PSCs in suspension culture while maintaining spheroid morphology and pluripotency of the expanded cells. Gibco Human Episomal iPSCs are described in this example however, the PSC medium and general protocol is effective in expansion of other iPSC and ESC lines. In this example, Essential 8 medium is used for initial 2D seeding post-cryopreservation. Other cell culture media are suitable for use in 2D and/or 3D seeding, including others known in the art and those provided herein.

Essential 8™ complete medium was prepared by thawing supplements overnight at 2 to 8° C. The thawed supplements were mixed by gentle inversion. 10 mL of the supplement mixture was transferred to 500 mL the Essential 8 basal medium. 5 mL of an antibiotic and antimycotic was added to the Essential 8 complete medium and mixed by gentle inversion. This complete medium can be stored for up to 2 weeks at 2-8° C., protected from light.

The PSC complete medium as described and provided herein was prepared by thawing supplements as described herein overnight at 2 to 8° C. or for about 1 hour at room temperature. The thawed supplements were mixed by gentle inversion. 100 mL of the PSC supplement mixture was transferred to 900 mL of PSC basal medium. 10 mL of an antibiotic and antimycotic was added to the PSC complete medium and mixed by gentle inversion. This complete medium can be stored for up to 2 weeks at 2-8° C., protected from light.

At Day 0 cryopreserved Gibco Human Episomal iPSCs were seeded on Geltrex coated 6-well plates. 20 mL of Essential 8™ complete medium and 200 μL of RevitaCell™ supplement were added to two 50 mL conical tubes and warmed to 37° C. in a water bath for no more than 5 minutes. 1.5 mL of the Essential 8 complete medium was added to each well of the plate. The iPSCs were resuspended in 2 mL of Essential 8 complete medium with RevitaCell. Countess II automated cell counter was used to count the resuspended cells. The cells were seeded at three cell seeding densities: 40,000 viable cells/cm², 60,000 viable cells/cm², and 80,000 viable cells/cm² in the wells. The seeded cells were incubated overnight in a 37±1° C., 5% (±1%) CO2 humidified incubator. On Days 1 and 2 after seeding the media in the wells was aspirated and cells were fed with 1 mL pre-warmed (37° C.) Essential 8 complete medium.

On Day 3 following seeding, the iPSCs were passaged. 2D Versene passage of the iPSCs was performed. The confluency of the 2D cultures was estimated. PSCs ideally should be between 50-80% confluent at passaging. 1 well was selected to passage into a new 2D plate. After incubation with Versene solution the cells were resuspended in 8 mL of the respective growth medium. The cell suspension was transferred to duplicate wells at 1:8, 1:12, and 1:16 split ratio and the plates were incubated at 37° C., 5% CO2 for about 24 hours. The following day, the 2D plates were fed with the Essential 8 complete medium. Feeding was performed daily. 2D plates were passaged with Versene whenever the iPSCs reached 50-80% confluency. 2D cultures were used to supply cells for 3D experiments. Non-tissue culture treated 6-well plates were used for 3D cultures. 13 μL of 10 mM Y-27632 was added to 13 mL aliquots of PSC complete media. 2 mL of the PSC complete medium+Y-27632 solution was added to each well of the 6-well plate. The plates were stored in a 37° C.+5% CO2 humidified incubator.

StemPro™ Accutase™ cell dissociation reagent or TrypLE™ Select enzyme passaging of 2D Gibco Human Episomal iPSCs was performed to seed 3D cultures. For example, the remaining wells of the 2D culture were used for Accutase passaging. 5 mL of each media condition was aliquoted into a 15 mL conical tube and 5 μL of 10 mM Y-27632 was added to each of the media aliquots. After incubation with the StemPro Accutase solution, the cells were resuspended in 1 mL of appropriate medium per well passaged by using the PSC complete medium+Y-27632. Countess II automated cell counter was used to count the resuspended cells. The cells were seeded into the wells at a seeding density of 300,000 viable cells/well. The plates were incubated overnight on an orbital shaker platform (rotated continuously at 70 RPM) in a 37° C.+5% CO2 humidified incubator.

On Days 4, 8, and 12, the iPSCs should be spheroids. Healthy spheroids typically appear rounded with a minimally “pitted” appearance whereas unhealthy or differentiating spheroids may appear misshapen and noticeably “pitted.” The spheroids begin to form with the first 24 hours of plating the 3D culture. The 3D spheroids were fed by replacing 50% of the medium with respective fresh pre-warmed medium. On Days 5, 9, and 13 the spheroids are not fed. On Days 6, 10, and 14 the spheroids are fed by replacing 50% of the medium with respective fresh pre-warmed medium. On Days 7, 11, and 15 the iPSC spheroids were passaged using Accutase. Non-tissue culture treated 6-well plates were used and 2 mL of complete PSC media with Y-27632 was added to each well. The spheroids from each well of the non-tissue culture treated 6-well plate were transferred to a tube, spin down, and incubated in 1 mL of pre-warmed StemPro Accutase. The cells were resuspended in 1 mL of appropriate medium by using the complete PSC medium+Y-27632. Countess II automated cell counter was used to count the resuspended cells. The cells were seeded into the wells at a seeding density of 300,000 viable cells/well. The plates were incubated overnight on an orbital shaker platform (rotated continuously at 70 RPM) in a 37° C.+5% CO2 humidified incubator. At the end of Passage 3, i.e., Day 15, cell count and viability were used to determine the fold change and percentage of viable cells.

In other culturing protocols, spheroids are passaged every 3-5 days. In some cases, the cells are seeded on day (as described) followed by a media overlay step on day 2, no feeding on day 3, feeding on day 4 and passaging on day 5.

To scale up from the 6-well format the appropriate culture conditions were determined for additional culture vessels. To adapt the 6-well format to a different culture vessel format, exemplary parameters of Table 2 were used.

TABLE 2
Parameters for scaling of PSC culture

| 3D Culture Format | Recommended RPM | Culture Volume |
|---|---|---|
| 6 well plate | 70-80 RPM | 2 mL per well |
| 12 well plate | 90-100 RPM | 1 mL per well |
| 24 well plate | 120-130 RPM | 500 μL per well |
| 48 well plate | 150-160 RPM | 250 μL per well |
| 125 mL shaker flask | 70 RPM | 20 mL |
| 250 mL shaker flask | 70 RPM | 40 mL |
| 100 mL PBS bioreactor | 40 RPM-70 RPM | 100 mL |
| 500 mL PBS bioreactor | 40 RPM | 500 mL |

### Example 2 A Glycogen Synthase Kinase-3 (GSK3) Inhibitor Improves Expansion of Pluripotent Stem Cells (PSC) and Maintains Morphology

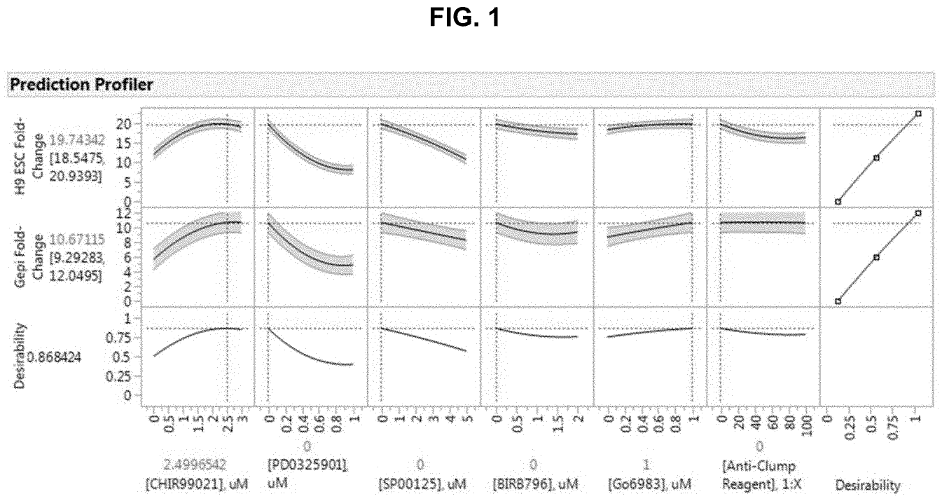

Previous naïve media formulations have been shown to require a rich, complex, media base containing, but not limited to, KSR, Albumax II, N2 Supplement, DMEM/F12, GlutaMAX, non-essential amino acids, insulin, and ascorbic acid. Addition of 4 or 5 small molecules and the growth factors LIF, TGFbeta1, and FGF to this base medium has been shown to support expansion of a more naive state of PSC in adherent and suspension culture (Gafni, et al., Nature 504(7479): 282-286 (2013); Lipsitz, et al., PNAS 115(25): 6369-6374 (2018)). Design of Experiment (DOE) was conducted using Gibco Human Episomal induced pluripotent stem cells (iPSCs) and WA09 embryonic stem cells (ESCs) to determine the impact of the small molecules. These PSCs were seeded in base PSC medium+1× RevitaCell™ Supplement and grown in a 24 well non-tissue culture treated plate at 120 RPM on an orbital shaker platform and cell expansion was assessed via viable cell counts on Day 5. Additionally, the impact of an anti-clump reagent was included as publications indicated that for the naïve media formulation assessed in Lipsitz, et al., PNAS 115(25): 6369-6374 (2018), inclusion of dextran sulfate aided in cell expansion by maintaining spheroids in a more consistent, smaller size (Lipsitz, et al., Biotechnol Bioeng. 115(8): 2061-2066 (2018)). FIG. 1 depicts results from the assessment of various concentrations of an anti-clumping agent and individual small molecules CHIR99021, PD0325901, SP00125, BIRD796, and Go6983on the expansion of iPSCs and WA09 hESCs. These data indicate that in the presence of our basal PSC medium formulation, only CHIR99021 is shown to provide a significant improvement in fold-expansion of PSCs. PD0325901 and SP00125 are shown to be detrimental to overall PSC expansion. No significant benefit was shown in the context of our formulation for the inclusion of anti-clumping reagent. CHIR99021 alone in the presence of the base PSC medium formulation surprisingly assisted in cell nucleation.

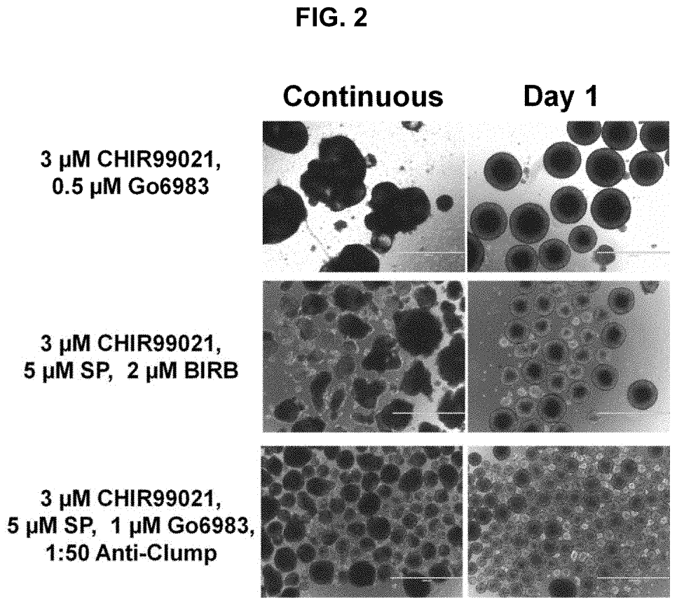

While addition of CHIR99021 was shown to improve the fold-change of cells in suspension culture, the spheroidal morphology was lost. Cell morphology was assessed following exposure to formulations containing 3 μM CHIR99021 in continuous culture media vs exposure at day 1 only. iPSCs were grown in a 24 well non-tissue culture treated plate at 120 RPM on an orbital shaker platform and cell morphology was assessed on Day 5. In this experiment, the small molecules were added on Day 1 only or throughout the culture period. Exemplary phase contrast micrographs of iPSCs grown in various conditions are shown in FIG. 2. While typical smooth edges and spheroid morphology was observed for conditions in which CHIR99021 was included only at the time of passaging (day 1), aberrant differentiation was noted for some conditions in which high concentrations of 3 μM CHIR99021 were included on a continual basis.

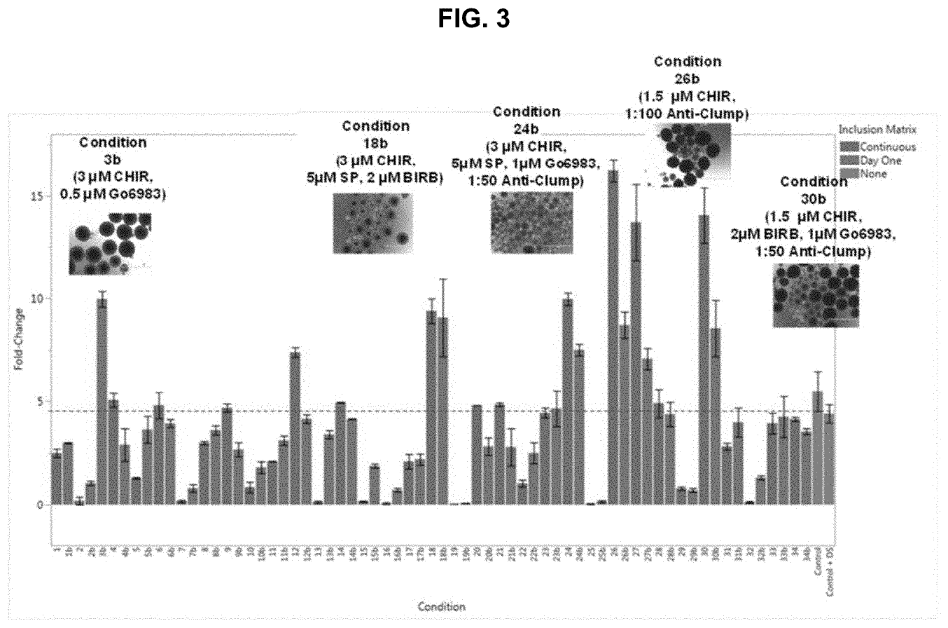

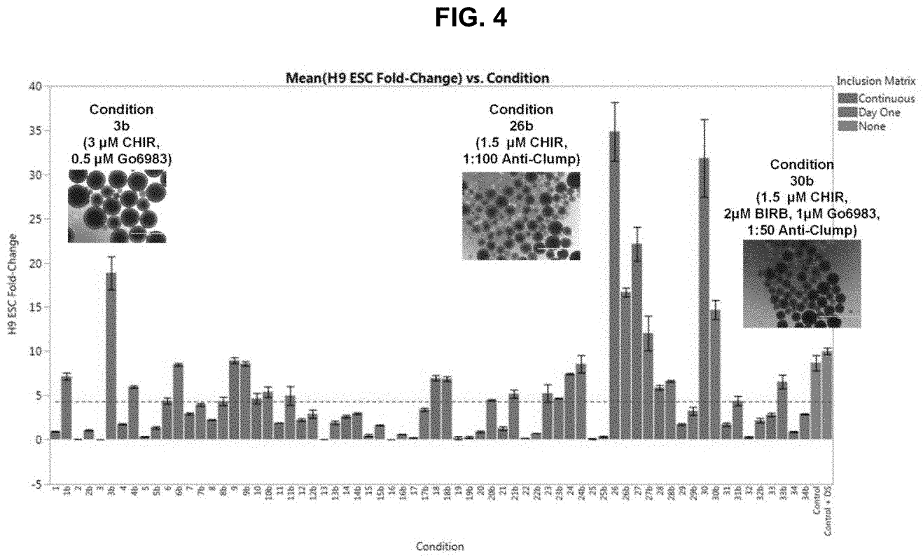

Cell expansion and morphology were assessed for PSC suspension cultures following incubation with the base PSC media formulation containing CHIR99021 alone or in combination with one or more of the other small molecules and/or anti-clumping agent. PSCs were grown in a 24 well non-tissue culture treated plate at 120 RPM on an orbital shaker platform and cell morphology was examined and cell expansion was assessed via viable cell counts on Day 5. In this experiment, the small molecules were either added on Day 1 only or throughout the culture period. Exemplary results using Gibco Human Episomal iPSCs are shown in FIG. 3 and using WA09 ESCs are shown in FIG. 4. In these figures, the x-axis indicates various formulations tested and the y-axis indicates the fold-change in viable cell counts from day 1 to day 5 of culture. Micrographs of the cell cultures at day 5 under certain conditions are also shown.

Shifting to addition of CHIR99021 on Day 1 only followed by complete base PSC medium only on Days 2-4 feeding was shown to support PSC expansion and maintain normal spheroidal morphology (FIG. 2-FIG. 4). It was surprising that the increase in fold-change could be accomplished through inclusion of CHIR99021 at the time of passage only (e.g., Day 1 only). These data indicate that inclusion of CHIR99021 at various concentrations only at the time of passaging is effective at increasing fold-expansion while minimizing unwanted aberrant differentiation as assessed via visual assessment of spheroidal morphology.

### Example 3 Determination of Rho Kinase Inhibitors (ROCKi) for Use with CHIR99021

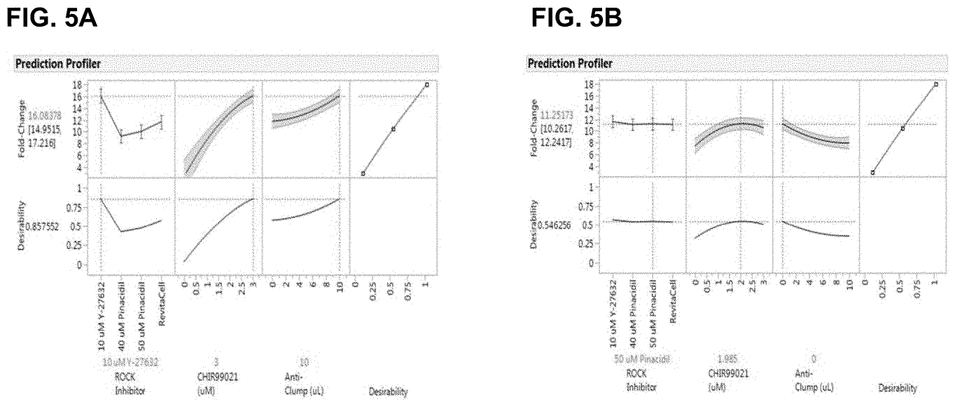

The experiments described in Example 2 were conducted in the presence of RevitaCell™ Supplement, a supplement containing a rho kinase inhibitor (ROCKi) coupled with molecules containing antioxidant and free radical scavenger properties. DOE was conducted using Gibco Human Episomal iPSCs to assess the impact of ROCKi/Cocktails Y-27632, Pinacidil, and RevitaCell™ Supplement on PSC cell survival and expansion in suspension culture when CHIR99021 or anti-clump reagent was added. For the assessment with results shown in FIG. 5, Y-27632 (10 μM), Pinacidil (40 μM), Pinacidil (50 μM) or RevitaCell™ supplement (1×) was included in PSC culture medium in the presence of CHIR99021 (3 μM) or 10 μL anti-clump reagent. PSCs were grown in a 24 well non-tissue culture treated plate at 120 RPM on an orbital shaker platform and cell expansion was assessed via viable cell counts on Day 5. In this experiment, the small molecules were added on Day 1 only.

Inclusion of ROCKi in conjunction with CHIR99021 at the time of passaging was shown to result in optimal fold-change with Y-27632 showing the greatest efficacy. (FIG. 5). Equivalent cell expansion with the other ROCKi/Cocktails was observed, yielding lower fold-change but not requiring the presence of anti-clump reagent (FIG. 5).

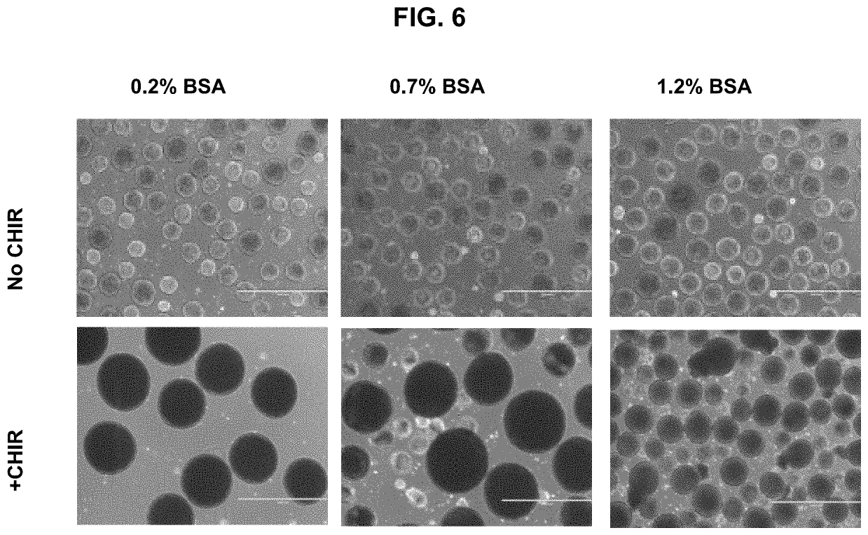

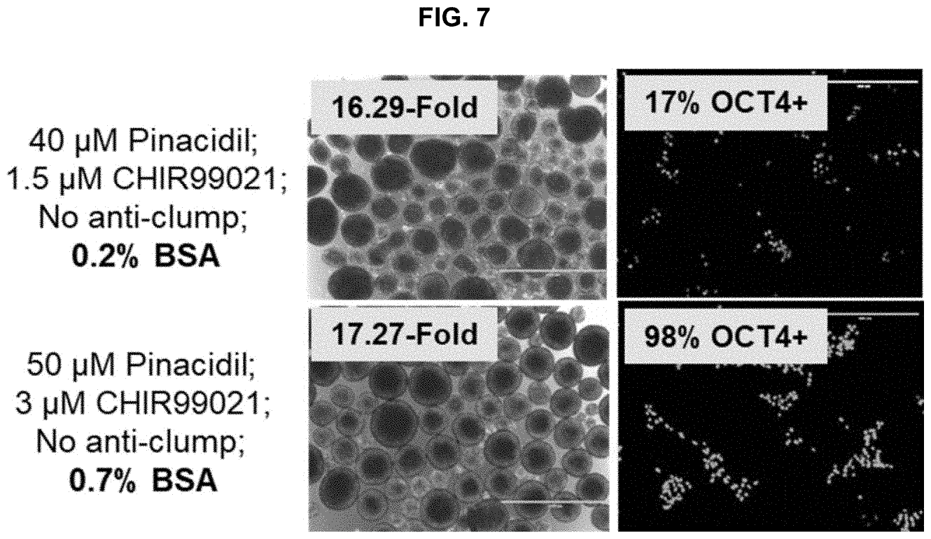

DOE was conducted to assess the balance of CHIR99021, Go6983 (a broad-spectrum PKC inhibitor), and BSA in achieving optimum fold-expansion, while also providing support of maintenance of pluripotency. PSCs were seeded in the presence of 10 μM Y-27632 and grown in suspension culture in PSC base medium containing various concentrations of BSA in the presence or absence of 3 μM CHIR99021. Variation in spheroid size resulting from the various culture conditions was assess by phase contrast imaging. When cells are seeded in the presence of 10 μM Y-27632, CHIR99021 aids in spheroid formation (FIG. 6). iPSCs were seeded in the presence of Pinacidil and grown in suspension culture in the presence of various concentrations of CHIR99021, BSA, and anti-clumping agent. At day 5 of culture spheroids were dissociated using StemPro Accutase and replated into 2D format for 24 hours. After 24 hours of growth, the cells were fixed, permeabilized and stained with DAPI and for Oct4 expression to determine the pluripotency of the dissociated cells. The number of cells expressing Oct4 was compared to the total number of cells expressing DAPI, enabling an estimation of the percentage of pluripotent stem cells obtained from the spheroids. Decreased pluripotency was observed at lower concentrations of BSA, indicating that a balance between CHIR99021 and albumin is required for maintenance of pluripotency (FIG. 7).

PSC expansion in suspension culture was also analyzed using human serum albumin (HSA) or recombinant HSA (rHSA) replacing BSA in the PSC medium formulation. Gibco Human Episomal iPSCs were expanded in non-tissue culture treated 6 well plates on an orbital shaker set to 70 RPM. The cells were passaged from 2D cultures using StemPro Accutase and grown in PSC medium with CHIR99021. Cells were seeded on Day 0 at a concentration of 150,000 cells/mL in a 2 mL volume, with a ROCK inhibitor added to the culture. Spheroids were grown over 5 days, with 50% medium replacement performed daily. After 5 days, an 8- to 12-fold expansion of iPSCs was obtained using HSA or rHSA in the PSC medium formulation containing CHIR99021.

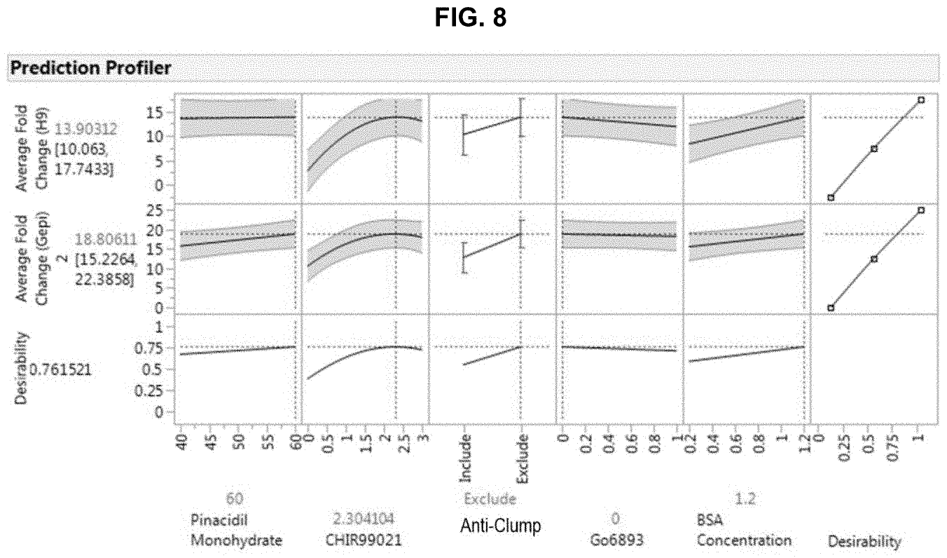

Various concentrations of Pinacidil, CHIR99021, Go6893, and BSA, with or without anti-clumping agent, was assessed for achieving fold-expansion of iPSCs in suspension culture. DOE prediction profiler analysis for determination of optimal concentrations, particularly focusing upon BSA and CHIR99021 as it relates to fold expansion, shows that an intermediate concentration of CHIR99021 and a high concentration of BSA provides maximal fold change (FIG. 8). Overall, CHIR99021 facilitates spheroid formation and a balance of albumin and CHIR99021 maintains appropriate spheroid size. Surprisingly, despite formulations having similar fold-changes, high concentrations of albumin are optimal for maintenance of pluripotency in the presence of CHIR99021. It was determined that across two cell lines (WA09 ESCs and Gibco Human Episomal iPSCs), exclusion of Go6983 and anti-clump agent is preferred with high levels of BSA balancing intermediate concentration of CHIR99021.

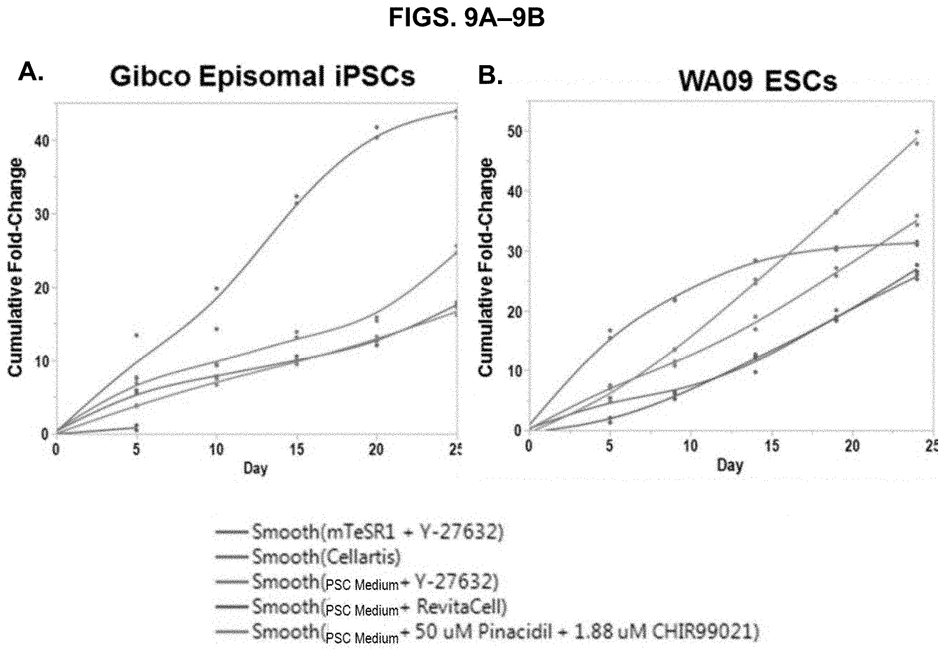

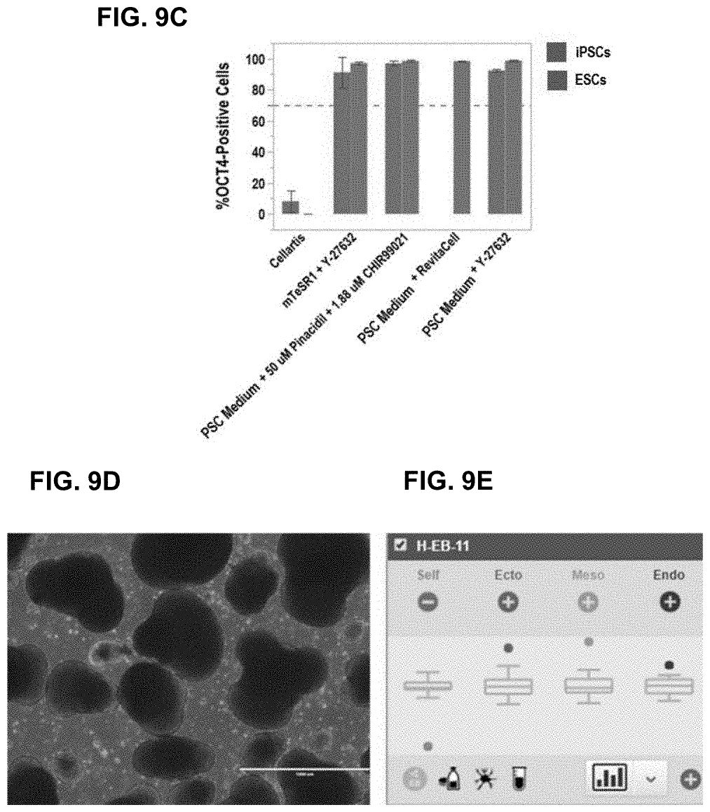

Gibco Human Episomal iPSCs and WA09 ESCs were grown in the following media conditions (FIGS. 9A-9B) in a 6-well non-tissue culture treated plate at 70 RPM on an orbital shaker platform with 50 percent medium replacement being performed daily: mTeSR1+Y-27632, Cellartis, Def-CS 3D PSC medium+Y-27632, PSC medium+RevitaCell™ supplement, PSC medium+50 μM Pinacidil+1.88 μM CHIR99021. Cell expansion was subsequently assessed via viable cell counts every 5 days, maintaining cells seeded at 150,000 viable cells/mL for 5 passages (FIGS. 9A-9B). In the context of utilization of Pinacidil together with intermediate concentrations of CHIR99021 at the time of passaging, fold-expansion in medium was shown to be comparable to for the iPSCs or improved for the ESCs relative to the mTeSR1 formulation. Performance when pairing with CHIR99021 was significantly improved for both cell lines relative to pairing with RevitaCell™ Supplement with larger gains observed for the ESCs and was comparable or improved over what was observed for mTeSR1+Y-27632 (FIGS. 9A-9B). Comparison of performance in terms of maintaining pluripotency following 3 passages of expansion was assessed via immunocytochemistry (ICC). The percentage of cells expressing OCT4 was assessed quantitatively using the Cellomics CX5 Cellinsight. The % OCT4 positive cells is highlighted for Gibco Human Episomal iPSCs (blue bars) and WA09 ESCs (red bars) (FIG. 9C). While Cellartis Def-CS 3D was shown to result in higher fold cell expansion for iPSCs than the PSC medium formulation provided herein, the cells expanded in Cellartis Def-CS 3D suffer from significant loss of pluripotency (FIG. 9C). Following 5 Days expansion, WA09 ESCs expanded in PSC medium+50 μM Pinacidil+1.88 μM CHIR99021 were transferred to Essential 6 Medium and allowed to spontaneously differentiate. Following 10 days in suspension culture in Essential 6 Medium the cells were assessed for trilineage differentiation potential using the TaqMan™ hPSC Scorecard™ Panel and were shown to efficiently differentiate to all three lineages (FIGS. 9D-9E). Cells expanded using our PSC Medium formulation containing CHIR99021 and passaged with Pinacidil were shown to maintain trilineage differentiation potential, a classic hallmark of pluripotent stem cells.

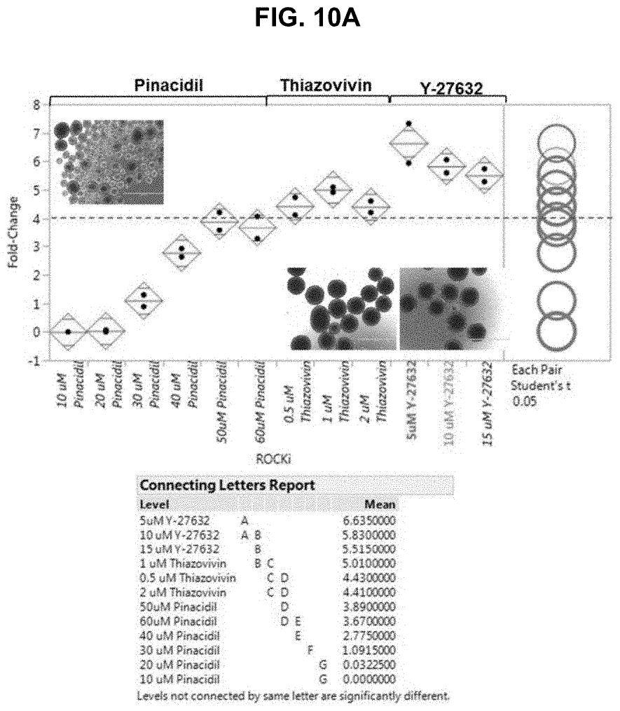

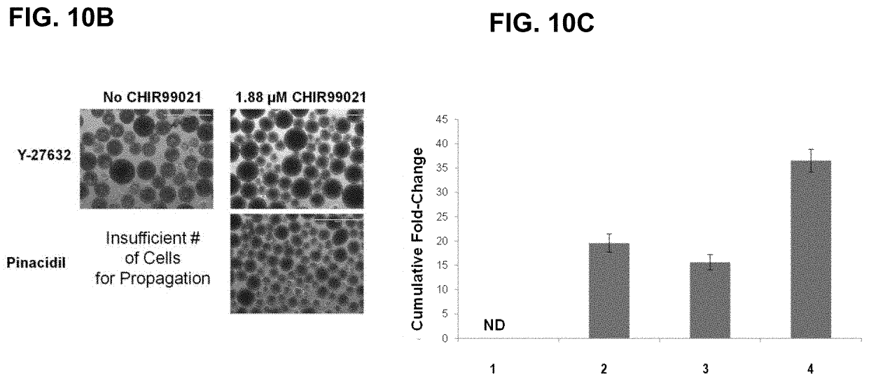

ROCK inhibitors Pinacidil, Thiazovivin, and Y-27632 were assessed for pairing with the PSC base medium formulation. Gibco Human Episomal iPSCs were seeded at 150,000 viable cells/mL into PSC base medium formulation with various concentrations of Pinacidil, Thiazovivin, or Y-27632. Following 5-day growth with 50% medium exchange daily and constant agitation on an orbital shake platform, fold-change upon expansion was assessed. FIG. 10A shows fold-change in iPSC expansion with the pairing of various concentrations of Pinacidil, Thiazovivin or Y-27632 with base PSC culture medium excluding CHIR99021. FIG. 10B shows phase contrast micrographs of iPSCs seeded with Y-27632 or Pinacidil and cultured in PSC media with or without CHIR99021. Larger spheroids were identified for conditions that used Y-27632 and Thiazovivin at the time of passaging relative to Pinacidil. Follow-up experiments with the PSC medium formulation confirmed pairing with Y-27632 with CHIR99021 for maximal fold-change as assessed using 3 passage cumulative fold-change (FIG. 10C) in expansion of iPSCs cultured with the following PSC media formulation: 1) PSC medium+1× RevitaCell, 2) PSC medium+1× RevitaCell+1.88 μM CHIR99021, 3) PSC medium+10 μM Y-27632, 4) PSC medium+10 μM Y-27632+1.88 μM CHIR99021. Y-27632 was shown to have the greatest impact on improving fold-change in the presence of the PSC Medium formulation when included only at the time of passage.

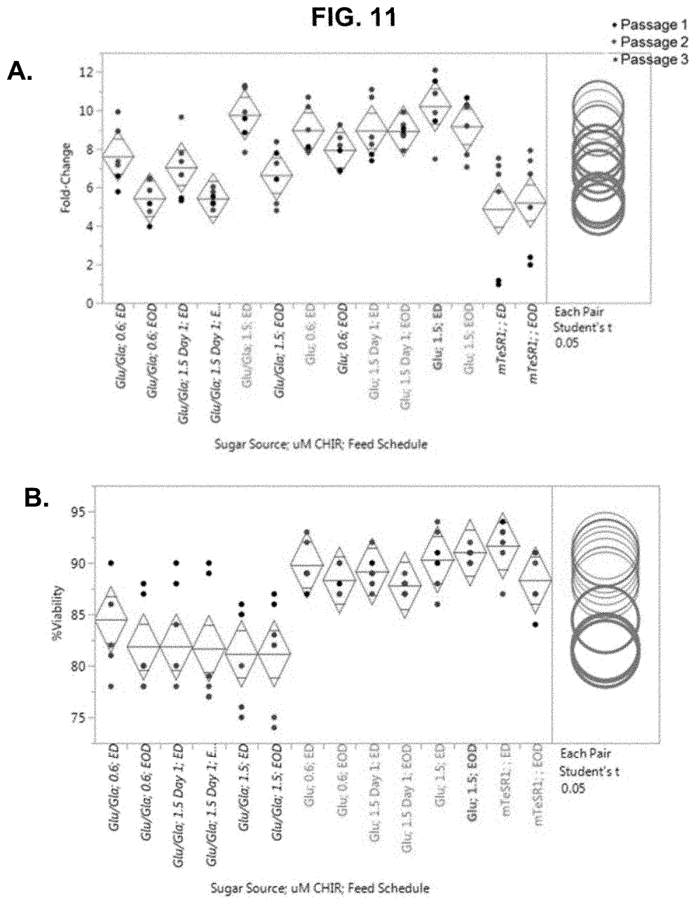

The optimal range of CHIR99021 for use in the PSC Medium formulation if included in the formulation for everyday feeds was assessed. Gibco Human Episomal iPSCs were expanded in 100 mL PBS bioreactors in duplicate and cell counts were performed at the end of each passage. Cells were seeded in 60 mLs of medium on Day 1 supplemented with 10 μM Y-27632, overlaid with 40 mLs of additional medium on Day 2, and then 50% medium exchange being completed every day (ED) or every-other-day (EOD). The sugar source (glucose/galactose) and the concentration of CHIR99021 was varied. The feed schedule was also modified to include every day vs. every-other-day feed schedules. Fold-change upon expansion and viability were assessed for each condition. CHIR99021 concentrations as low as 0.6 μM supported optimal cell expansion on an every-day medium exchange feed schedule (FIG. 11A) and viability on an every-day and every-other-day medium exchange feed schedule (FIG. 11B). Low viability was observed for glucose/galactose containing formulations at later passages and difficult to passage clusters resulting in decreased viability and resultant fold-change. From these data it was observed that CHIR99021 concentrations as low as 0.6 μM continuously included in the medium provided optimal fold-change relative to commercially available mTeSR1 medium formulation. Concentrations up to 1.5 μM CHIR99021 were shown to support optimal fold-expansion even when cultures were fed on an every-other-day cadence. (FIGS. 11A-11B).

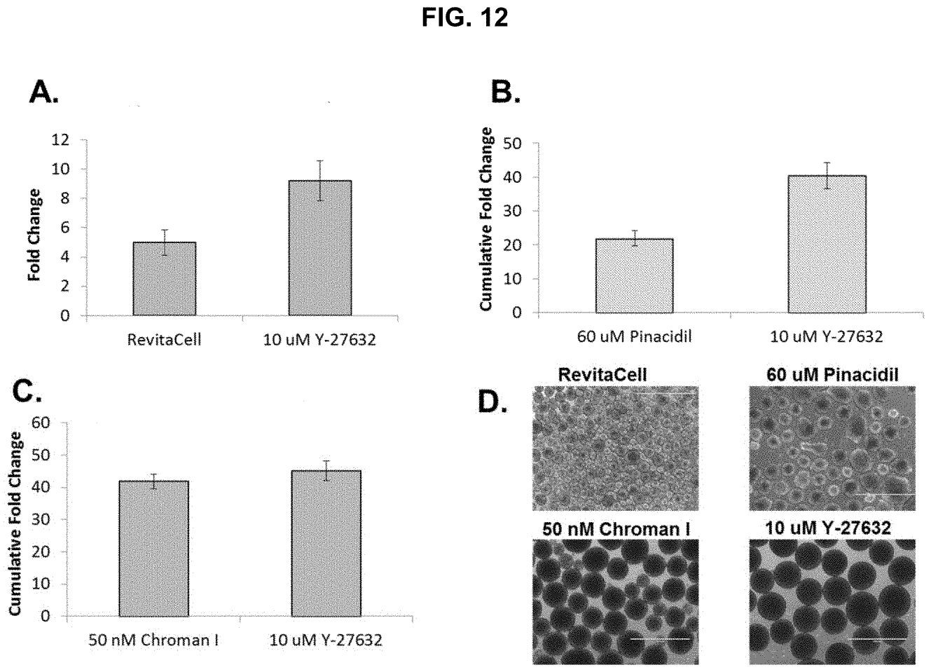

ROCKi Chroman I was compared to Y-27632, Pinacidil, and RevitaCell in the PSC medium for PSC suspension culture. Gibco Human Episomal iPSCs were expanded in non-tissue culture treated 6 well plates on an orbital shaker set to 70 RPM. The cells were passaged from 2D cultures using StemPro Accutase and grown in PSC Medium. Cells were seeded on Day 0 at a concentration of 150,000 cells/mL in a 2 mL volume, with a ROCK inhibitor added to the culture. The ROCKi added was either 1× RevitaCell (FIG. 12A), 60 μM Pinacidil (FIG. 12B), or 50 μM Chroman I (FIG. 12C), with all compared against 10 μM Y-27632. Spheroids were grown over 5 days, with 50% medium replacement performed daily. RevitaCell and 60 μM Pinacidil perform similarly and were inferior to 10 μM Y-27632 when expanding spheroids (FIGS. 12A-12B). 50 nM Chroman I and 10 μM Y-27632 perform similarly when expanding spheroids (FIG. 12C). RevitaCell and 60 μM Pinacidil spheroids are morphologically smaller, while 50 nM Chroman I and 10 μM Y-27632 spheroids are morphologically larger (FIG. 12D). Overall, optimal performance in the context of the CHIR99021 containing PSC media formulation was shown for Y-27632 and Chroman 1 passaged cultures.

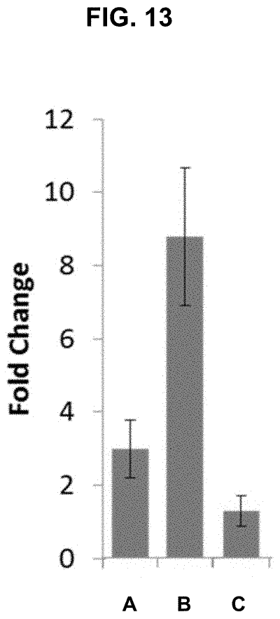

PSC expansion using the PSC media formulations provided herein was compared to expansion in commercially available media. H9 (WA09) ESCs were expanded in PSC basal medium in the absence of CHIR99021, PSC basal medium in the presence of CHIR99021, and in the mTeSR1 media formulation (Stem Cell Technologies). PSCs were passaged using StemPro™ Accutase™ cell dissociation reagent and grown in 6 well non-tissue culture treated plates seeded at 150,000 viable cells/mL in 2 mL of media and agitated at 70 RPM on an orbital shaker platform. For these comparisons, 10 μM Y-27632 was added to the cell cultures at the time of passaging to promote spheroid nucleation. Cell expansion was assessed via viable cell counts on Day 5. Cultures were fed with 50% medium replacement performed daily. As shown in FIG. 13, the addition of CHIR99021 to PSC basal medium enhances fold-expansion compared to PSC basal medium without CHIR99021 FIG. 13B vs. FIG. 13A) and significantly enhanced fold-expansion compared to mTeSR1 (FIG. 13C).

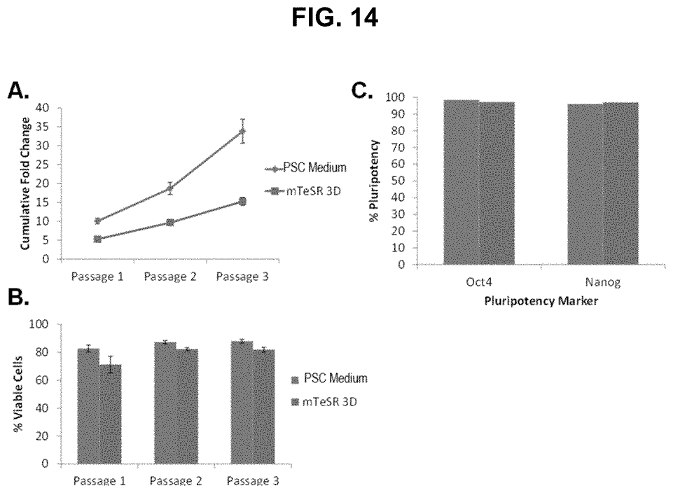

Gibco Episomal iPSCs were expanded in non-tissue culture treated 6 well plates on an orbital shaker set to 70 RPM. The cells were passaged from 2D cultures using StemPro Accutase and grown in complete PSC medium or mTeSR-3D medium. Complete PSC medium cultures were fed by 50% medium replacement daily, whereas mTeSR-3D medium cultures were fed using the manufacturer recommended overlay protocol by addition of 224 μL feed medium daily. Cultures were assessed for fold-expansion over 3 passages (FIG. 14A), maintenance of viability over the course of passaging (FIG. 14B), and maintenance of pluripotency as assessed using flow cytometry (FIG. 14C) for Oct4 and Nanog expression. Overall, PSC complete medium formulation was shown to have improved fold-change over mTeSR-3D while maintaining comparable percent pluripotency relative to mTeSR-3D.

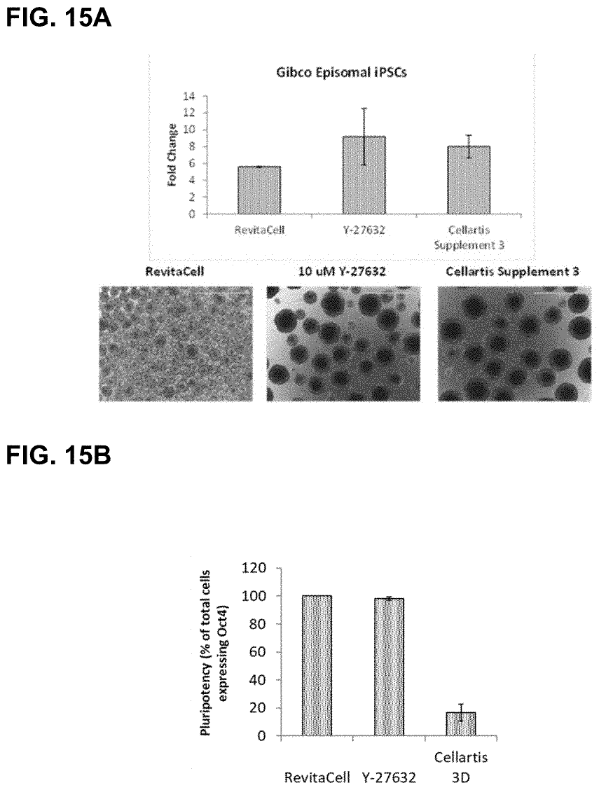

PSC fold-expansion and pluripotency maintenance was compared for spheroid cultures seeded with a ROCK inhibitor or with the Cellartis Supplement 3 of the commercially available Cellartis Def-CS 3D medium system and grown in the complete PSC medium provided herein. Gibco Human Episomal iPSCs were expanded in 24-well non tissue culture treated plates at 120 RPM on an orbital shaker platform in the complete PSC medium to determine whether the type of ROCKi or Supplement selected for use affects the overall spheroid growth. All PSCs were grown in the complete PSC medium formulation, with the only exception being on day 1, the cells were grown either in RevitaCell (1×), Y-27632 (10 μM), or Cellartis Supplement 3 (500× dilution). Overall spheroid growth and expansion was assessed after the cells had grown for 5 days in the 24 well plate (FIG. 15A). The pluripotency of the spheroids was also assessed after the cells had grown for 5 days in the 24 well plate (FIG. 15B). To assess pluripotency, spheroids were dissociated using StemPro Accutase and replated into 2D format for 24 hours. After 24 hours of growth, the cells were fixed, permeabilized and stained with DAPI and for Oct4 expression to determine the pluripotency of the dissociated cells. The number of cells expressing Oct4 was compared to the total number of cells expressing DAPI, enabling an estimation of the percentage of pluripotent stem cells obtained from the spheroids. Y-27632 was shown to be the most efficient with larger spheroids on Day 5 being grown in cultures where Y-27632 is added at the time of passaging vs. RevitaCell™ Supplement. The Cellartis Supplement 3 did have a comparable size and morphology relative to Y-27632. RevitaCell and Y-27632 when used in conjunction with the PSC media formulation can maintain high levels of pluripotent stem cells during spheroid growth. Cellartis Def-CS 3D, in contrast to complete PSC medium, was insufficient to maintain pluripotency of the cells as indicated by the decrease in OCT4 expression in the expanded cell population (FIG. 15B).

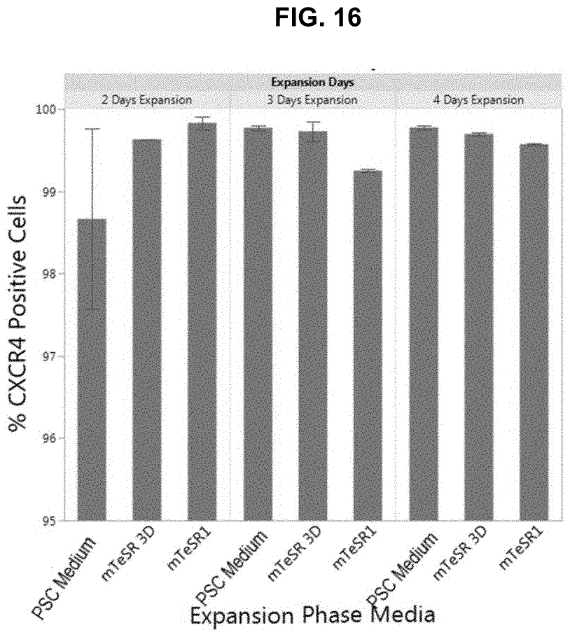

While expansion of pluripotent stem cells is an important first step in a PSC suspension culture workflow, it is also important that following expansion, cells can undergo directed differentiation to the desired downstream lineage. To assess downstream differentiation capability, PSCs were seeded into various media conditions in 6 well non-tissue culture treated plates and expanded at 70 RPM on an orbital shaker platform with 50 percent medium replacement being performed daily for 2, 3, or 4 days of expansion. After the expansion phase, definitive endoderm differentiation was conducted using the Gibco PSC Definitive Endoderm Induction Kit according to the kit protocol. Comparison of performance in terms of expression of CXCR4, a definitive endoderm marker, following the differentiation protocol was assessed quantitatively by flow cytometry using the Invitrogen Attune NxT flow cytometer. The percentage of Gibco Human Epsiomal iPSCs positive for CXCR4 is shown in the graph of FIG. 16. These indicate the ability of cells expanded using complete PSC medium formulation to undergo directed differentiation to the endodermal lineage. The endodermal differentiation performance with the PSC medium is comparable to what is observed with commercially available mTeSR1 and mTeSR3D media.

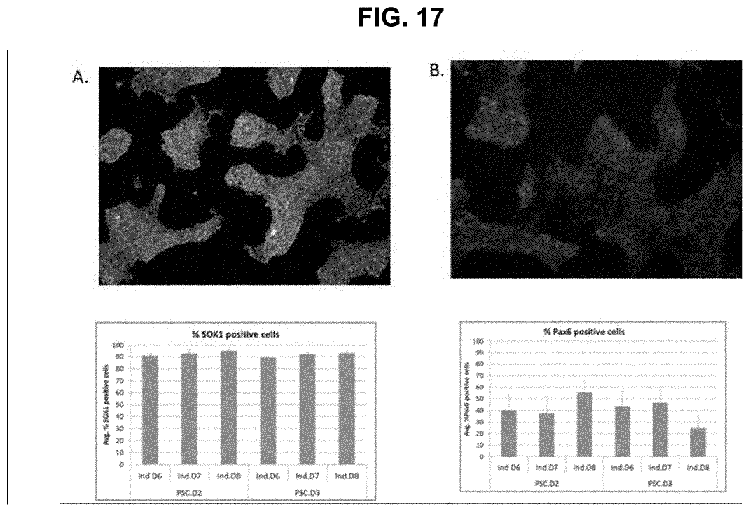

This evaluation was extended to assessment of the ability to differentiate to ectodermal lineages. Gibco Human Episomal iPSCs were cultured in complete PSC suspension culture medium for 2 or 3 days (PSC.D2, PSC.D3 in FIG. 17) for sphere formation. Medium was changed to PSC Neural Induction Medium for days 6, 7, and 8 days (Ind.D6, Ind.D7, Ind.D8 in FIG. 17). After induction, neural stem cell aggregates were cultured in expansion medium for 1 to 3 days. At day 11, cells were dissociated with Accutase and plated in monolayer on Geltrex coated plates in expansion medium for 2 days. Cells were fixed and stained for markers SOX1 and Nestin or PAX6 and OTx2. FIG. 17A depicts representative immunocytochemistry images of the expression of SOX1 (red) and Nestin (green) with quantitative expression of SOX1 shown in graph. FIG. 17B depicts representative ICC images of the expression of PAX6 (red) and OTx2 (green) with quantitative expression of PAX 6 shown in the graph. These data indicate the ability of cells expanded using complete PSC Medium to undergo directed differentiation using the Gibco Neural Induction Medium to the ectodermal lineage. Cell expansion and directed differentiation has also been shown to the mesodermal lineage.

### Example 4 Scalability of PSC Medium

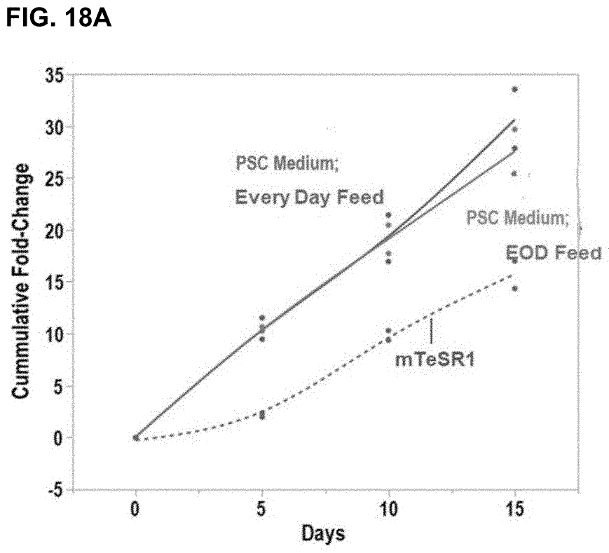

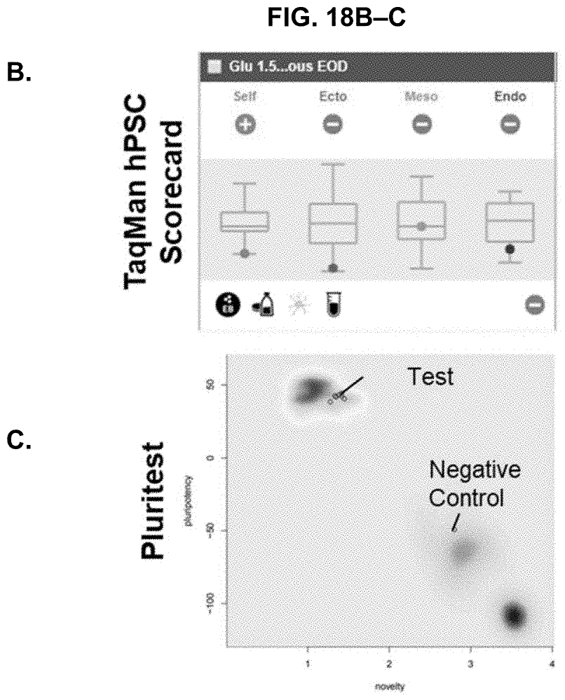

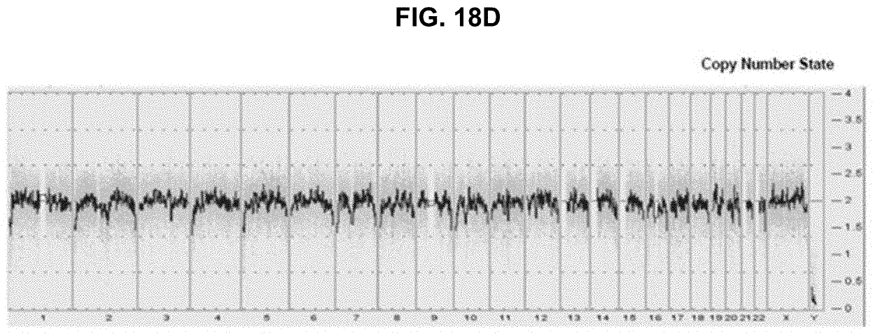

In addition to using 24 well and 6 well format suspension cultures, PSC expansion was evaluated in higher culture volumes. For this, expansion of PSCs in PSC Medium was assessed on 100 mL scale using PBS bioreactors. Gibco Human Episomal iPSCs were passaged using StemPro™ Accutase™ cell dissociation reagent and grown in 100 mL PBS mini bioreactors stirring at 40 rpm. Cells were cultured in PSC complete medium with continuous exposure to 1.5 μM CHIR99021 or with mTeSR1 medium. 10 μM Y-27632 was added only at the time of passaging. For the PSC medium cultures, the cells were fed using 50% medium replacement either every day (ED) or every-other-day (EOD). For the mTeSR1 condition, cells were fed daily using 50% medium replacement daily. FIG. 18A shows the fold-expansion of Gibco Human Episomal iPSCs over 3 passages for all conditions evaluated. Comparable expansion was observed for the PSC Medium formulation using every-other-day and every day feed schedules. Increased performance relative to mTeSR1 was observed at the 100 ml scale. Maintenance of normal pluripotent stem cell properties was assessed for the iPSC culture in PSC medium fed using the EOD 50% medium exchange schedule included: maintenance of pluripotency was assessed using the TaqMan™ hPSC Scorecard™ Assay (FIG. 18B) and PluriTest™ assay analysis (FIG. 18C), and maintenance of normal karyotype was assessed via KaryoStat analysis (FIG. 18D). In addition, Gibco Human Episomal iPSCs expanded under every-other-day PSC complete medium conditions were shown to maintain normal pluripotent stem cell properties, including maintenance of expression of pluripotency markers and maintenance of normal karyotype. Use of the provided PSC media and cell expansion workflow maintained the pluripotency of iPSCs and ESCs (WA09) grown as spheroids over 5 consecutive passages as assessed by PluriTest™ analysis and by flow cytometric analysis showing >90% of the same expanded cell lines expressing Oct4 and Nanog markers.

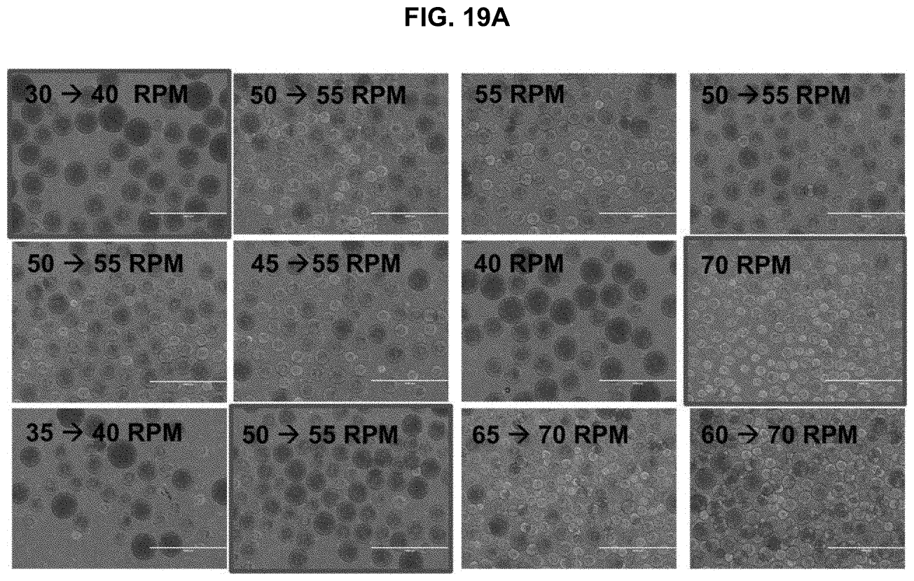

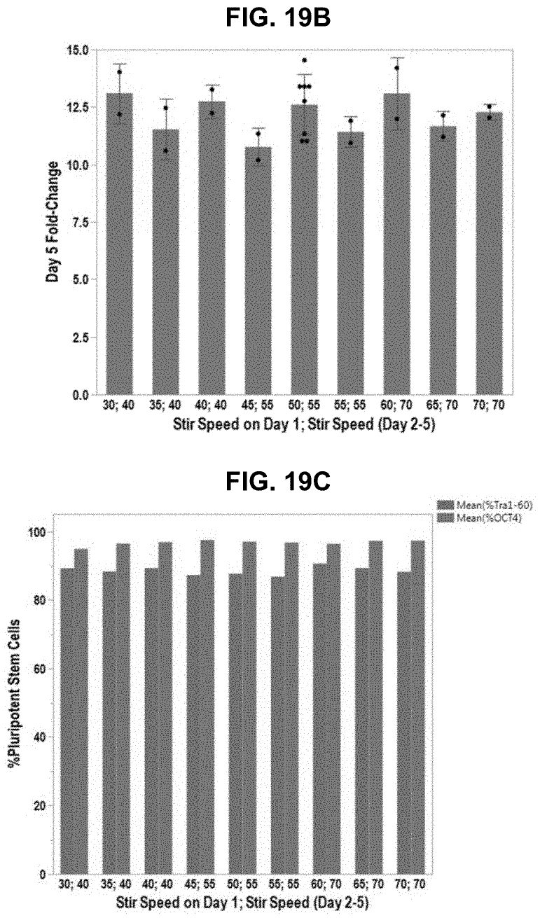

It has been shown that the size of spheroids obtained upon expansion can have an impact on the downstream differentiation capability when cells are initiated in suspension post-passaging and subsequently differentiated in suspension. To address this, the ability of cells to be “tuned” in spheroid diameter using the complete PSC Medium formulation was examined in conjunction with PBS mini Bioreactors at 100 mL scale by using a range of stir speeds. Gibco Human Episomal iPSCs were passaged using StemPro Accutase and grown in 100 mL PBS mini bioreactors, stirring at an initial stir speed for the first 24 hours post-passaging, followed by adjustment to a 2nd stir speed for Day 2-5. Cells were seeded into 60 mL of complete PSC medium supplemented with 10 μM Y-27632 at the time of passaging, overlayed with 40 mL of additional complete PSC medium alone on Day 2, followed by feeding every-other-day via 50% medium exchange. Assessment of spheroid morphology on Day 3 post-initiation indicated the presence of tunable spheroids with smaller spheroid diameter being achieved at higher RPMs (FIG. 19A). FIG. 19B depicts the fold-change assessed on Day 5 post-passage with cell counts from each individual flask being indicated by an individual data point. The stir speed conditions are indicated on the x-axis. Maintenance of pluripotency was assessed using flow cytometry to detect the presence of extracellular (TRA1-60, blue) and intracellular (OCT4, red) markers of pluripotency (FIG. 19C). These data indicate that the spheroid diameter can be modified using variable stir speeds. Comparable fold-change and pluripotency was observed on Day 5, regardless of the stir speeds used (e.g., 30-40 RPM).

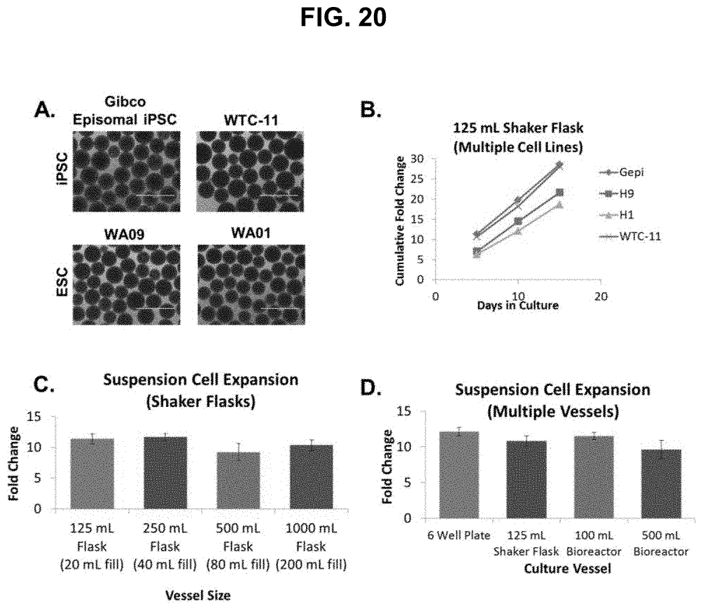

Spheroid growth in shaker flasks was analyzed. Gibco Human Episomal iPSCs, WTC-11 iPSCs, WA09 ESCs, and WA01 ESCs were expanded in shaker flasks (125 mL; 250 mL; 500 mL; 1000 mL) on an orbital shaker set to 70 RPM. The cells were passaged from 2D cultures using StemPro Accutase and grown in complete PSC medium. Cells were seeded on Day 0 at a concentration of 150,000 cells/mL in a 2 mL volume, with inhibitor Y-27632 added to the culture. Spheroids were grown over 5 days, with 50% medium replacement performed daily. The growth of multiple cell lines was compared. All four cell lines tested expanded as spheroids in PSC medium (FIGS. 20A-20B). Surprisingly, the PSC medium formulation provided an efficient solution for expansion of not only iPSCs, but also ESCs as demonstrated by the 4 cell lines cultured and expanded here, some of which do not easily transition into suspension culture. Over ten different iPSC and ESC cell lines have been shown to be compatible with suspension culture expansion using the provided PSC media and expansion workflow. On average, a 5-10 fold expansion per passage has been obtained and the fold expansion can vary by cell line. The performance of Gibco Human Episomal iPSCs in various shaker flask volumes (FIG. 20C) and in multiple types of vessels (FIG. 20D) was analyzed. All shaker flask sizes tested showed consistent expansion performance with the PSC medium. Shaker flask performance was similar to performance in 6 well plates and bioreactors.

1. A pluripotent stem cell (PSC) composition comprising:
a cell culture basal medium;
one or more mitogenic growth factors;
a glycogen synthase kinase-3 (GSK3) inhibitor, and
a rho kinase (ROCK) inhibitor,
wherein the composition provides one or more of enhancing PSC growth, enhancing PSC proliferation, maintaining PSC pluripotency, maintaining spheroid morphology, maintaining PSC morphology, increasing PSC passage count, or increasing PSC culture scale, as compared to customary PSC media.

2-5. (canceled)

6. The composition of claim 1, wherein the GSK3 inhibitor is selected from the group consisting of: CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), BIO ((2′Z,3′E)-6-bromoindirubin-3′-oxime), AR-A 014418 (N-[(4-methoxyphenyl)methyl]-N-(5-nitro-2-thiazolyl)urea)0, Kenpaullone (9-bromo-7,12-dihydro-indolo[3,2-d][1]benzazepin-6(5H)-one), SB 216763 (dichlorophenyl)-4-(1-methyl-1H-indol-3-yl)-1H-pyrrole-2,5-dione), or SB 415286 (3-[(3-chloro-4-hydroxyphenyl)amino]-4-(2-nitrophenyl)-1H-pyrrole-2,5-dione), their salts, their esters, and combinations thereof.

7. (canceled)

8. The composition of claim 1, wherein the ROCK inhibitor is selected from the group consisting of: Y-27632 ((R)-(+)-trans-4-(1-aminoethyl)-N-(4-pyridyl)cyclohexanecarboxamide), Chroman 1, Emricasan, Polyamines, Trans-ISRIB, thiazovavin, and combinations thereof.

9-12. (canceled)

13. The composition of claim 1, wherein the one or more mitogenic growth factors comprises EGF, TGF-α, TGF-β, bFGF, BDNF, HGF, heregulin (HRG), or KGF, or combinations thereof.

14. The composition of claim 1, wherein the composition comprises at least two mitogenic growth factors and wherein the at least two mitogenic growth factors comprise: EGF and TGF-α, EGF and TGF-β, EGF and bFGF, EGF and BDNF, EGF and HGF, EGF and HRG, EGF and KGF, TGF-α and TGF-β, TGF-α and bFGF, TGF-α and BDNF, TGF-α and HGF, TGF-α and HRG, TGF-α and KGF, TGF-β and bFGF, TGF-β and BDNF, TGF-β and HGF, TGF-β and HRG, TGF-β and KGF, bFGF and BDNF, bFGF and HGF, bFGF and HRG, bFGF and KGF, BDNF and HGF, BDNF and HRG, BDNF and KGF, HGF and HRG, HGF and KGF, or HRG and KGF.

15. The composition of claim 1, wherein the composition comprises albumin or peptides thereof, wherein the albumin is selected from the group consisting of human serum albumin, bovine serum albumin, rat serum albumin, mouse serum albumin, horse serum albumin monkey serum albumin, pig serum albumin, a recombinant serum albumin, functional fragments of any of the foregoing, and combinations thereof.

16. The composition of claim 1, wherein the composition further comprises one or more extracellular matrix (ECM) components.

17. (canceled)

18. The composition of claim 1, wherein the composition further comprises an induced pluripotent stem cell (iPSC) or an embryonic stem cell (ESC).

19-22. (canceled)

23. The composition of claim 1 further comprising insulin and/or transferrin.

24. (canceled)

25. A method for growing pluripotent stem cells (PSCs) in suspension, the method comprising:
providing PSCs to be cultured in suspension; and
contacting the PSCs with a first composition in a culture vessel to form a suspension culture, the first composition comprising:
(a) a cell culture basal medium;
(b) a first small molecular inhibitor, comprising a glycogen synthase kinase-3 (GSK3) inhibitor, salts thereof, or esters thereof,
(c) a second small molecule inhibitor, comprising a rho kinase (ROCK) inhibitor, salts thereof, or esters thereof,
(d) one or more mitogenic growth factors; and
optionally (e) one or more albumins or peptides thereof, and
culturing the suspension culture under conditions favorable for PSC expansion.

26-29. (canceled)

30. The method of claim 25, wherein the contacting is at a seeding step for the suspension culture.

31. The method of claim 30, wherein the seeding step is at passaging of the suspension culture.

32. The method of claim 30, further comprising contacting the PSCs with a second composition comprising (a), (b), (d) and (e) at least one day after the seeding or the passaging step to modify the suspension culture medium; and culturing the suspension culture in the presence of the second composition.

33-39. (canceled)

40. The method of claim 30, further comprising contacting the PSCs with a second composition comprising (a), (d) and (e) at least one day after the seeding or the passaging step to modify the suspension culture medium; and culturing the suspension culture in the presence of the second composition.

41-50. (canceled)

51. The method of claim 25, wherein the GSK3 inhibitor is selected from the group consisting of: CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), BIO ((2′Z,3′E)-6-bromoindirubin-3′-oxime), AR-A 014418 (N-[(4-methoxyphenyl)methyl]-N-(5-nitro-2-thiazolyl)urea)0, Kenpaullone (9-bromo-7,12-dihydro-indolo[3,2-d][1]benzazepin-6(5H)-one), SB 216763 (dichlorophenyl)-4-(1-methyl-1H-indol-3-yl)-1H-pyrrole-2,5-dione), or SB 415286 (3-[(3-chloro-4-hydroxyphenyl)amino]-4-(2-nitrophenyl)-1H-pyrrole-2,5-dione), their salts, their esters, and combinations thereof.

52. (canceled)

53. The method of claim 25, wherein the ROCK inhibitor is selected from the group consisting of: Y-27632 ((R)-(+)-trans-4-(1-aminoethyl)-N-(4-pyridyl)cyclohexanecarboxamide), Chroman 1, Emricasan, Polyamines, Trans-ISRIB, thiazovavin, and combinations thereof.

54. (canceled)

55. The method of claim 25, wherein the ROCK inhibitor inhibits ROCK1 activity and/or ROCK2 activity.

56-57. (canceled)

58. The method of claim 25, wherein the one or more mitogenic growth factors comprises EGF, TGF-α, TGF-β, bFGF, BDNF, HGF, heregulin (HRG), or KGF, or combinations thereof.

59. The method of claim 25, wherein the first composition comprises at least two mitogenic growth factors and wherein the at least two mitogenic growth factors comprise: EGF and TGF-α, EGF and TGF-β, EGF and bFGF, EGF and BDNF, EGF and HGF, EGF and HRG, EGF and KGF, TGF-α and TGF-β, TGF-α and bFGF, TGF-α and BDNF, TGF-α and HGF, TGF-α and HRG, TGF-α and KGF, TGF-β and bFGF, TGF-β and BDNF, TGF-β and HGF, TGF-β and HRG, TGF-β and KGF, bFGF and BDNF, bFGF and HGF, bFGF and HRG, bFGF and KGF, BDNF and HGF, BDNF and HRG, BDNF and KGF, HGF and HRG, HGF and KGF, or HRG and KGF.

60. The method of claim 25, wherein the first composition comprises albumin or peptides thereof, wherein the albumin is selected from the group consisting of human serum albumin, bovine serum albumin, rat serum albumin, mouse serum albumin, horse serum albumin monkey serum albumin, pig serum albumin, a recombinant serum albumin, functional fragments of any of the foregoing, and combinations thereof.

61. The method of claim 25, wherein the first composition further comprises one or more extracellular matrix (ECM) components.

62-65. (canceled)

66. The method of claim 25, further comprising agitating the cells while culturing the suspension culture in the first and second compositions.

67-68. (canceled)
